(19) United States
(12) Patent Application Publication
(10) Pub. No.: US 2022/0211990 A1
(43) Pub. Date: Jul. 7, 2022

(54) SYSTEM AND METHOD FOR FACILITATING CONTROL OF AND INTERACTION WITH A TATTOO MACHINE AND ASSOCIATED ACCESSORIES

(71) Applicant: FK Irons Inc., Doral, FL (US)

(72) Inventors: Gaston Siciliano, Doral, FL (US); Alan Lisler, Doral, FL (US)

(73) Assignee: FK Irons Inc., Doral, FL (US)

(21) Appl. No.: 17/571,090

(22) Filed: Jan. 7, 2022

Related U.S. Application Data
(60) Provisional application No. 63/134,848, filed on Jan. 7, 2021.
(60) Provisional application No. 63/151,375, filed on Feb. 19, 2021.
(60) Provisional application No. 63/155,051, filed on Mar. 1, 2021.

(51) Int. Cl. A61M 37/00; H04W 4/30; H04W 76/14

(52) U.S. Cl. CPC A61M 37/0076; H04W 4/30; H04W 76/14; A61M 2205/82; A61M 2205/502; A61M 2205/80; A61M 2205/35

(57) ABSTRACT

A system for facilitating control of and interaction with a tattoo machine and associated accessories is disclosed. In particular, the system includes a software application, which may be utilized to scan for devices within a communication range of a device executing the software application. Devices that are detected by the device and application may be wirelessly paired and/or synchronized with the application and with each other using any suitable communications technology. The detected devices, for example, may include wireless tattoo machines and associated tattoo machine accessories. Once the detected devices and/or application are wirelessly paired with each other, the system may enable a user to control the tattoo machine, the tattoo machine accessory, or both by utilizing the application. For example, the system may utilize the application to adjust settings of the tattoo machine, record tattoo machine usage, schedule tattoo sessions, and conduct a variety of other actions.

### CROSS REFERENCE TO RELATED APPLICATION

This application claims priority to and the benefit of U.S. Provisional Patent Application Ser. Nos. 63/134,848, filed on Jan. 7, 2021, 63/151,375, filed Feb. 19, 2021 and 63/155,051, filed Mar. 1, 2021, which are hereby incorporated by reference in their entireties.

### FIELD OF THE INVENTION

The present application relates to tattoo machine technologies, interconnectivity technologies, wireless communication technologies, accessory technologies, controller technologies, interaction technologies, and more particularly, to a system and method for facilitating control and interaction with a tattoo machine and associated accessories.

### BACKGROUND

In today's society, companies and individuals are increasingly spending time, financial resources, and efforts to develop technologies to meet the steadily increasing demand for tattoos. In order to apply a tattoo onto a person, tattoo artists have a variety of tattoo equipment at their disposal. For example, a variety of tattoo machines exist to facilitate the application of tattoo ink onto the skin of a person. Such machines include, but are not limited to, coil-based tattoo machines, rotary-based tattoo machines, air-based tattoo machines, pneumatic-based tattoo machines, among other machines. Certain machines may be modular in that a plurality of different components may be attached to the body of such machines depending on the specific use-case scenario that the tattoo artist is faced with. For example, different types of cartridges housing different types of tattoo needles may be secured to such machines depending on the person receiving the tattoo, the type of tattoo being applied, and other factors. Additionally, certain tattoo machines may be accessorized and/or fitted with foot pedals to facilitate control of the tattoo machine, varying types of power supplies, varying types of grips, varying types of inks, and other accessories. In addition to having the required talent, dexterity, and skill to design and apply a tattoo, tattoo artists also have to ensure that the tattoo machines and accessories are clean and sterile and that no cross-contamination between people being tattooed occurs.

Notably, while currently existing technologies provide convenience and various benefits, such technologies still come with various drawbacks and inefficiencies. For example, certain tattoo machines are constructed in such a way that they are unwieldy for tattoo artists and often accelerate tattoo artist fatigue. Additionally, existing tattoo machines often have multiple wires connecting the tattoo machines with its various accessories. This often makes such tattoo machines cumbersome and difficult to handle, particularly when such wires become entangled. Furthermore, existing tattoo machines often have limited settings for adjusting the operation of the tattoo machines, limited modularity with accessories, and limited configurations. Moreover, existing tattoo machines provide little to no status information about the components of the tattoo machines, the operational state of the tattoo machines, or the ability to communicate with other devices. Based on the foregoing, current tattoo machine-related technologies may be improved and enhanced so as to provide robust communication capabilities, increased tattoo machine modularity, useful tattoo machine status information, enhanced tattoo machine accessorizing capabilities, enhanced control capabilities, enhanced tattoo machine component interactions, and increased ease-of-use for tattoo artists.

### SUMMARY

A system and accompanying methods for facilitating control of and interactions with a tattoo machine and associated accessories are disclosed. In particular, the system and methods enable a user to create an account with an application providing the operative functionality of the present disclosure. The system and methods may enable the user to enter various preferences, demographic information, user-related information, and other information in a user profile associated with the account. Once the user is authenticated into the account, such as by utilizing authentication credentials, the system and methods may enable the user to access the functionality provided by the application. In particular, the application may provide the user with the option to scan for devices within a communication range of a user device of the user that is executing or otherwise allowing the user to utilize the application. Once the option is selected via the application, the system and methods may include enabling the user device of the user to scan for the devices within the communication range of the user device of the user. Based on the scanning conducted by the device, the system and methods may include detecting tattoo machines, tattoo machine accessories, and/or other devices within the communication range of the user device. Once the tattoo machines, tattoo machine accessories, and/or other devices are detected, the system and methods may provide options, via the application, to pair any combination of the detected devices with the user device and/or the application executing on the device. Once the desired pairing option is selected, the system and methods may facilitate the pairing of various combinations of tattoo machines and tattoo machine accessories using the user device and/or application.

When devices are paired with the user device and/or the application, the system and methods may include visually rendering an indication via the user interface of the application indicating which devices have been paired and/or synchronized with each other. The system and methods may then allow the user to control the operation of any one or more of the paired devices, such as by voice commands, digital input commands, options presented in the user interface of the application, or through other methodologies. For example, the system and methods may enable the user to activate and/or deactivate a paired device, track usage statistics of a paired device, adjust settings of a paired device and/or its components, unpair certain devices from one another, and/or conduct a variety of other control operations. The system and methods may also allow for, such as via the application, the booking and scheduling of tattoo sessions with potential tattoo clients, the providing of digital release authorizations to allow a user to consent to being tattooed, tattoo session timers, the ability to photograph a tattoo, the ability to upload media content corresponding to a tattoo to be tattooed on an individual, detection of component failures and/or problems of the paired devices, tracking whether supplies associated with the tattoo machines and/or tattoo machine accessories need to be replenished, ordering replacement supplies, ordering tattoo machines and/or tattoo machine accessories, scan an image or media content of interest (such as for inspiration for a tattoo), receiving of feedback from the user and/or other users, posting content to social media or online platforms, and/or a myriad of other functionality. Once the user is done controlling and/or interacting with the paired devices, the user may unpair any of the paired devices and log out of the user's account. When the user wants to control the devices again, the user may simply repeat the processes facilitated by the system and methods as desired.

In one embodiment, a system for facilitating control of and interaction with a tattoo machine and associated accessories is provided. The system may include a memory that stores instructions and a processor that executes the instructions to perform various operations of the system. The system may perform an operation that includes enabling a user to create a user account with a software application for controlling and/or interacting with tattoo machines, tattoo machine accessories, or a combination thereof. Once the user is authenticated into the software application using the created account, the system may perform an operation that includes providing, via a user interface of the software application, an option to scan for devices within a communication range of a device associated with the application. In certain embodiments, the devices may include tattoo machines, tattoo machine accessories, any other devices, or a combination thereof. After the option is selected via the software application, the system may perform an operation that includes scanning for the devices within the communication range of the device associated with the application. Based on the scanning, the system may perform an operation that includes detecting the tattoo machines, the tattoo machine accessories, or a combination thereof, within the communication range of the device associated with the application. Based on the user's preferences input into the application, the system may perform an operation that includes pair the device and/or the application with the detected tattoo machines, tattoo machine accessories, or a combination thereof. Once the desired pairings are achieved, the system may perform an operation that includes enabling the device to control the tattoo machines, the tattoo machine accessories, or a combination thereof, by utilizing the application.

In another embodiment, a method for facilitating control of and interaction with a tattoo machine and associated accessories is disclosed. The method may include a memory that stores instructions and a processor that executes the instructions to perform the functionality of the method. In particular, the method may include providing, via a user interface of an application, an option to scan for devices within a communication range of a device associated with the application. In certain embodiments, the devices may include tattoo machines, tattoo machine accessories, or a combination thereof. After the option is selected, the method may include scanning for the devices within the communication range of the device associated with the application. Based on the scanning, the method may include detecting the tattoo machines, the tattoo machine accessories, or a combination thereof, within the communication range of the device associated with the application. The method may proceed to include pairing the device and/or application with the tattoo machines and/or tattoo machine accessories. In certain embodiments, the pairing may also include pairing the tattoo machines and/or tattoo machines accessories with each other. After pairing the device and/or application with the tattoo machines, the tattoo machine accessories, or a combination thereof, the method may include utilizing the device to control the tattoo machines and/or the tattoo machine accessories by utilizing the application.

According to yet another embodiment, a computer-readable device comprising instructions, which, when loaded and executed by a processor cause the processor to perform operations, for facilitating control of and interaction with tattoo machines and/or tattoo machine accessories, the operations comprising: providing, via a user interface of an application, an option to scan for devices within a communication range of a device utilizing the application, wherein the devices comprise a tattoo machine, a tattoo machine accessory, or a combination thereof; scanning, after the option is selected, for the devices within the communication range of the device utilizing the application; detecting, based on the scanning, the tattoo machine, the tattoo machine accessory, or both, within the communication range of the device utilizing the application; pairing the device with the tattoo machine, the tattoo machine accessory, or both; and enabling, after pairing the device with the tattoo machine, the tattoo machine accessory, or both, the device to control the tattoo machine, the tattoo machine accessory or both, by utilizing the application.

These and other features of the systems and methods for facilitating control of and interaction with a tattoo machine and associated accessories are described in the following detailed description, drawings, and appended claims.

### BRIEF DESCRIPTION OF THE DRAWINGS

FIG. 1 is a schematic diagram of a system for facilitating control and interaction with a tattoo machine and associated accessories according to an embodiment of the present disclosure.

FIG. 2 is a sample user-interface of an application for use with the system of FIG. 1 according to an embodiment of the present disclosure.

FIG. 3 is a sample use-interface of an application for use with the system of FIG. 1, which illustrates a user account creation option and login capabilities of the application.

FIG. 4 is a sample user-interface of an application for use with the system of FIG. 1, which enables a user to create an account with the application.

FIG. 5 is a sample user-interface of an application for use with the system of FIG. 1, which enables a user to retrieve an account via entry of an email associated with the user's account.

FIG. 6 is a sample user-interface of an application for use with the system of FIG. 1, which facilitates the scanning of devices within communication range of the device executing the application.

FIG. 7 is a sample user-interface of an application for use with the system of FIG. 1, which enables the pairing of devices that have been scanned and detected by the device executing the application.

FIG. 8 is a sample user-interface of an application for use with the system of FIG. 1, which illustrates a visual indication of when devices are paired using the application.

FIG. 9 is a sample user-interface of an application for use with the system of FIG. 1, which selection of a preferred language via a settings features of the application.

FIG. 10 is a sample user-interface of an application for use with the system of FIG. 1, which provides a rendering of a tattoo machine accessory and its corresponding voltage, settings for adjusting voltage, settings for activating the accessory and/or tattoo machine, and e-give settings.

FIG. 11 is a sample user-interface of an application for use with the system of FIG. 1, which enables the tattoo machine and/or an accessory of the tattoo machine to be controlled using voice commands.

FIG. 12 is a sample user-interface of an application for use with the system of FIG. 1, which provides a visual indication when a voice command capability is active.

FIG. 13 is a sample user-interface of an application for use with the system of FIG. 1, which provides a rendering of another tattoo machine accessory and its corresponding voltage, settings for adjusting voltage, settings for activating the accessory, a battery level indicator, and e-give settings.

FIG. 14 is a sample user-interface of an application for use with the system of FIG. 1, which enables voice command activation for another tattoo machine accessory.

FIG. 15 is a sample user-interface of an application for use with the system of FIG. 1, which provides a visual indication when the voice command capability for an accessory is active.

FIG. 16 is a sample user-interface of an application for use with the system of FIG. 1, which provides a rendering of another tattoo machine accessory and its corresponding voltage, settings for adjusting voltage, settings for activating the accessory, a battery level indicator, and e-give settings.

FIG. 17 is a sample user-interface of an application for use with the system of FIG. 1, which enables voice command activation for another tattoo machine accessory.

FIG. 18 is a sample user-interface of an application for use with the system of FIG. 1, which provides a visual indication when the voice command capability for an accessory is active.

FIG. 19 is a sample user-interface of an application for use with the system of FIG. 1, which enables synchronization of any number of devices associated with the tattoo machine.

FIG. 20 is a sample user-interface of an application for use with the system of FIG. 1, which enables the purchase of a tattoo machine and/or accessory associated with the tattoo machine.

FIG. 21 is a sample user-interface of an application for use with the system of FIG. 1, which enables synchronization of any number of devices associated with the tattoo machine.

FIG. 22 is a sample user-interface of an application for use with the system of FIG. 1, which enables the setting of the e-give level for a tattoo machine.

FIG. 23 is a sample user-interface of an application for use with the system of FIG. 1, which provides a plurality of help options available to a user of the application.

FIG. 24 is a sample user-interface of an application for use with the system of FIG. 1, which provides access to a plurality of tutorials for enabling a user to utilize the tattoo machine and/or accessories.

FIG. 25 is a sample user-interface of an application for use with the system of FIG. 1, which enables a firmware update for a tattoo machine and/or associated accessories.

FIG. 26 is a sample user-interface of an application for use with the system of FIG. 1, which enables a user to select a device, enable voice control, and adjust e-give settings.

FIG. 27 is a sample user-interface of an application for use with the system of FIG. 1, which enables a firmware update for a wireless battery pack.

FIG. 28 is a sample user-interface of an application for use with the system of FIG. 1, which enables a firmware update for a tattoo machine.

FIG. 29 is a flow diagram illustrating a sample method for facilitating control and interaction with a tattoo machine and associated accessories according to an embodiment of the present disclosure.

FIG. 30 is a schematic diagram of a machine in the form of a computer system within which a set of instructions, when executed, may cause the machine to facilitate control and interaction with a tattoo machine and associated accessories.

### DETAILED DESCRIPTION OF THE DRAWINGS

A system 100 and accompanying methods for facilitating control and operation of a tattoo machine and associated tattoo machine accessories are disclosed. In particular, the system 100 and methods enable a user, such as first user 101, to generate an account with a software application providing the operative functionality of the present disclosure. The system 100 and methods may enable the user to include various preferences, demographic information, user-related information, desired settings, and other information in a user profile of the account. Once the user authenticates into the account via the application, the system 100 and methods may enable the user to access the functionality provided by the application. For example, the application may provide the user with the option to scan for devices within a communication range of a user device (e.g. first user device 102) of the user that is executing or otherwise allowing the user to utilize the application. Once the option is selected by the user via the application, the system 100 and methods may include enabling the user device of the user to scan for the devices within the communication range of the user device of the user. Based on the scanning conducted by the device, the system and methods may include detecting tattoo machines (e.g. tattoo machine 120), tattoo machine accessories (e.g. wireless battery pack 122, hover device 124, footswitch 126, etc.), and/or other devices within the communication range of the user device. Once the tattoo machines, tattoo machine accessories, and/or other devices are detected based on the scanning, the system 100 and methods may provide options, via the application, to pair any combination of the detected devices with the user device and/or the application executing on the device. Once the desired pairing option is selected by the user via the user interface of the application, the system 100 and methods may facilitate the pairing of various combinations of tattoo machines and tattoo machine accessories using the user device and/or application.

When devices are paired with the user device and/or the application, the system 100 and methods may include visually rendering a confirmation of the pairing via the user interface of the application. The system 100 and methods may then allow the user to control the operation of any one or more of the paired devices, such as by voice commands, digital input commands, options presented in the user interface of the application, or through other techniques. For example, the system 200 and methods may enable the user to activate and/or deactivate a paired device, track usage statistics of a paired device, adjust settings of a paired device and/or its components, unpair certain devices from one another, and/or conduct a variety of other control operations. The system 100 and methods may also facilitate and enable the booking and scheduling of tattoo sessions, the providing of digital release authorizations to allow a user to consent to being tattooed and to assume any possible risks of tattooing, tattoo session timers, the ability to photograph a tattoo, the ability to upload media content corresponding to a tattoo to be tattooed on an individual, detection of component failures and/or problems of the paired devices, tracking whether supplies associated with the tattoo machines and/or tattoo machine accessories need to be replenished, ordering replacement supplies, ordering tattoo machines and/or tattoo machine accessories, scanning an image or media content of interest (such as for inspiration for a tattoo), receiving of feedback from the user and/or other users, posting content to social media or online platforms, and/or a myriad of other functionality. Once the user is done controlling and/or interacting with the paired devices, the system 100 and methods may enable the user to unpair any of the paired devices using the application and may log out of the application. When the user wants to control the devices at a future time, the user may log into the application again and repeat the aforementioned processes using the functionality provided by the system and methods.

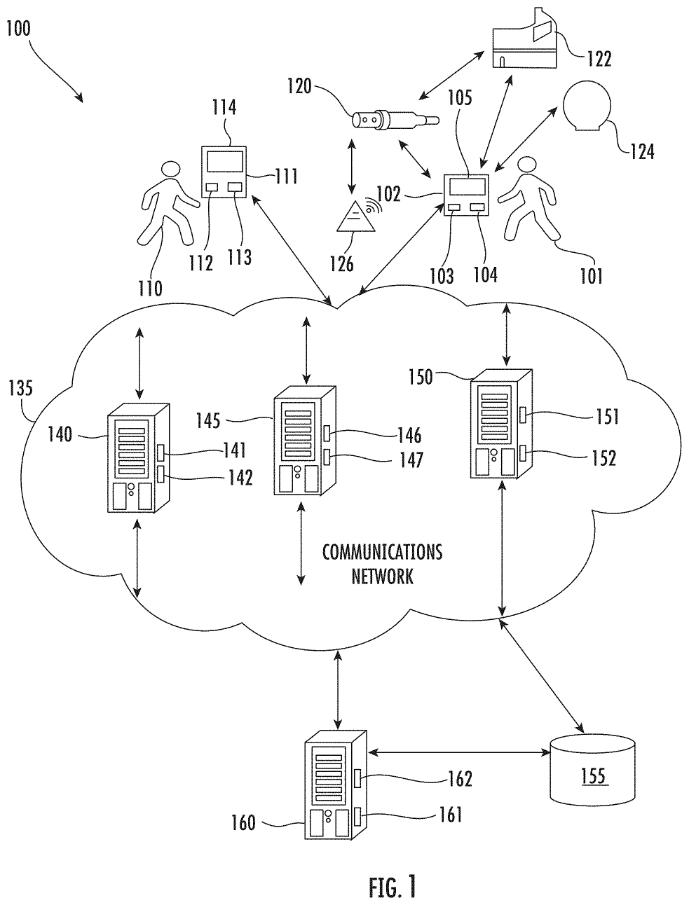

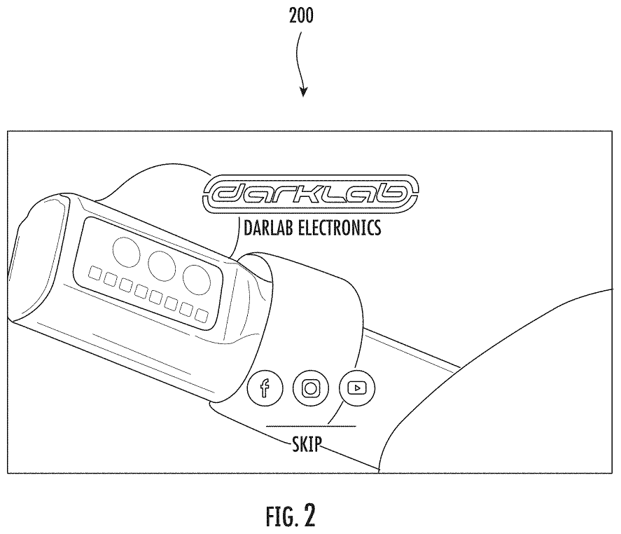

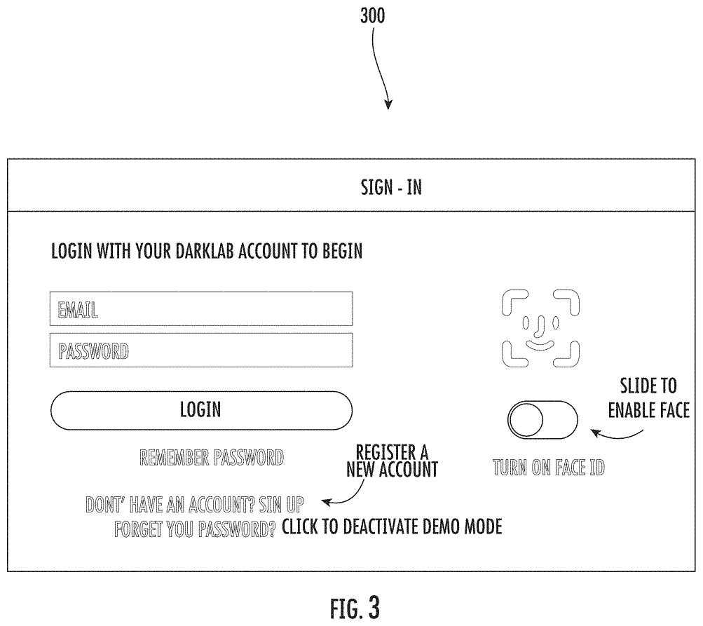

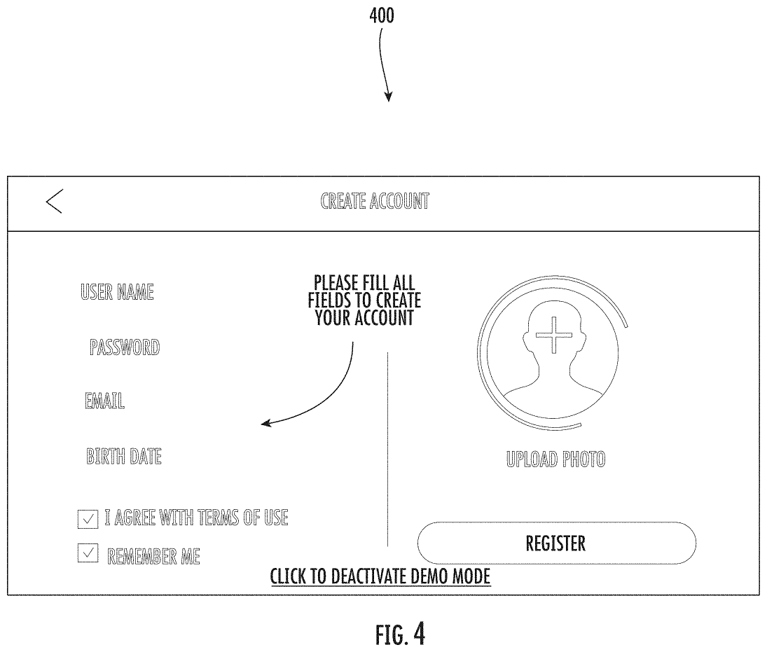

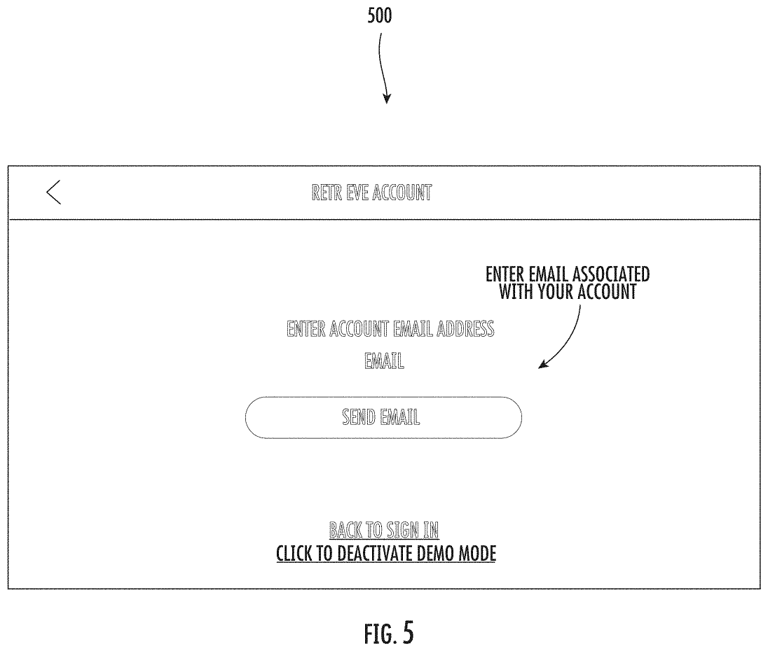

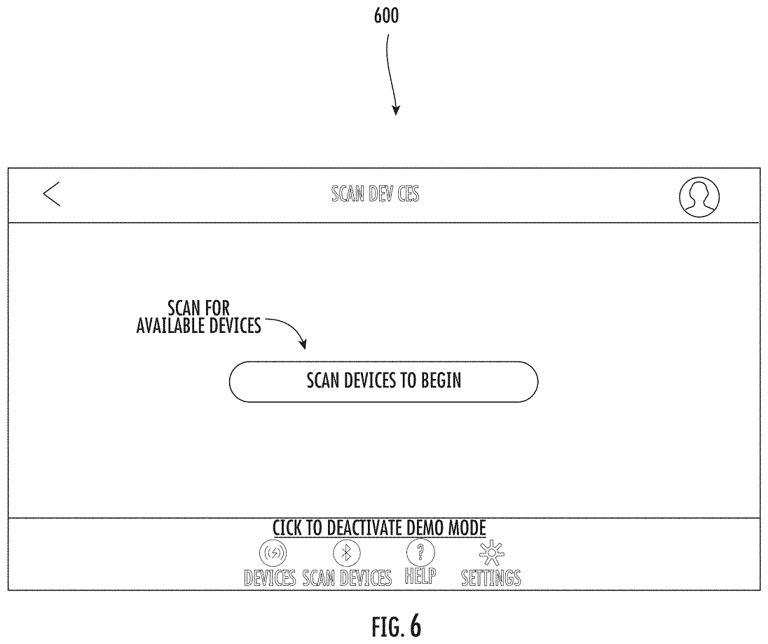

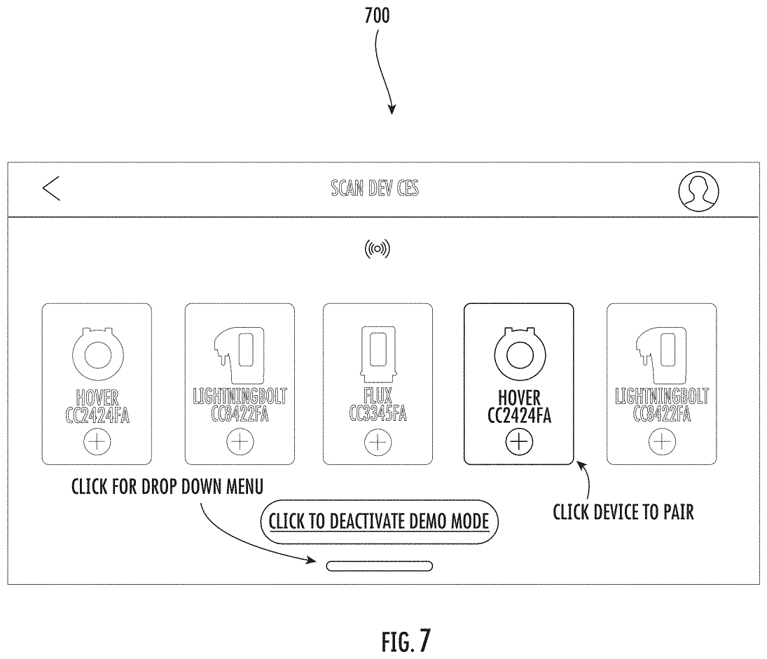

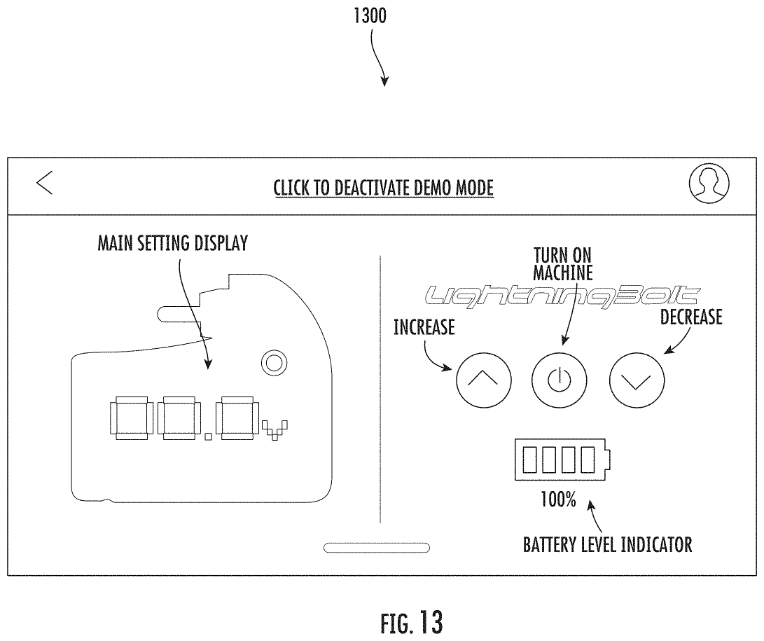

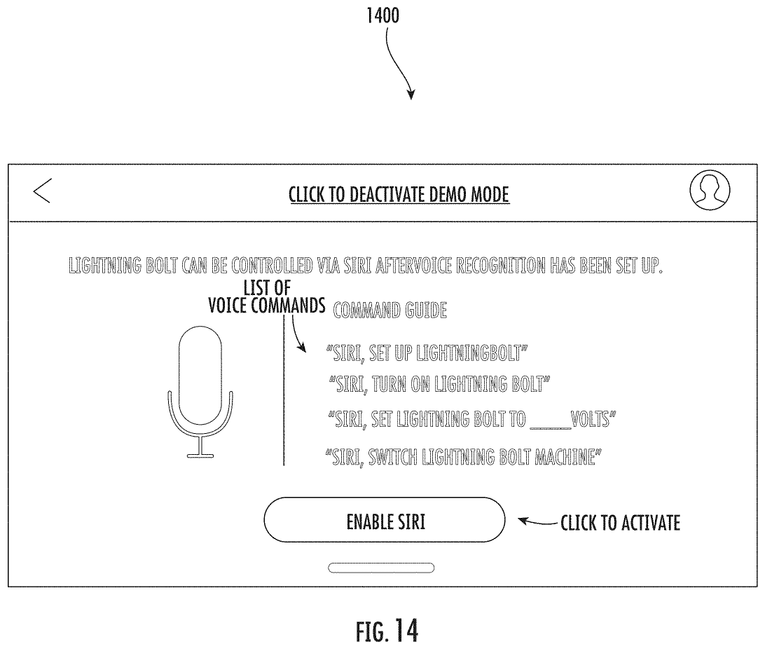

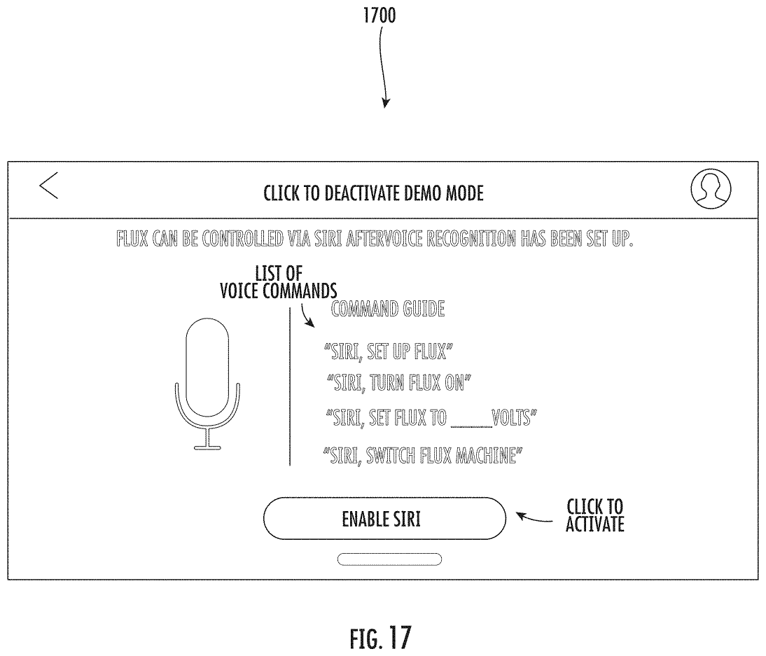

As shown in FIG. 1 and referring also to FIGS. 2-30, a system 100 for facilitating control and operation of a tattoo machine and associated tattoo machine accessories is disclosed. Notably, the system 100 may be configured to support, but is not limited to supporting, tattoo machine systems and services, tattoo machine accessory systems and services, device pairing protocols, services, and technologies, artificial intelligence services and systems, machine learning services and systems, content delivery services, monitoring systems and services, cloud computing services, satellite services, telephone services, voice-over-internet protocol services (VoIP), software as a service (SaaS) applications, platform as a service (PaaS) applications, gaming applications and services, social media applications and services, product ordering applications and services, operations management applications and services, productivity applications and services, mobile applications and services, and/or any other computing applications and services. Notably, the system 100 may include a first user 101, who may utilize a first user device 102 to access data, content, and services, or to perform a variety of other tasks and functions. As an example, the first user 101 may utilize first user device 102 to transmit signals to access various online services and content, such as those available on an internet, on other devices, and/or on various computing systems. As another example, the first user device 102 may be utilized to access an application that provides the operative functions of the system. In certain embodiments, the first user 101 may be a tattoo artist, a tattoo recipient or client, a robot, a humanoid, a program, a computer, any type of user, or a combination thereof. The first user device 102 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, etc.) that may enable the first user 101 to interact with various applications executing on the first user device 102 and to interact with the system 100. In certain embodiments, the first user device 102 may be and/or may include a computer, any type of sensor, a laptop, a set-top-box, a tablet device, a phablet, a server, a mobile device, a smartphone, a smart watch, and/or any other type of computing device. Illustratively, the first user device 102 is shown as a smartphone device in FIG. 1 and the first user 101 is a tattoo artist. In certain embodiments, the first user device 102 may be utilized by the first user 101 to control the operative functionality of the system 100.

In addition to using first user device 102, the first user 101 may also utilize and/or have access to additional user devices. As with first user device 102, the first user 101 may utilize the additional user devices to transmit signals to access various online services and content. The additional user devices may include memories that include instructions, and processors that executes the instructions from the memories to perform the various operations that are performed by the additional user devices. In certain embodiments, the processors of the additional user devices may be hardware, software, or a combination thereof. The additional user devices may also include interfaces that may enable the first user 101 to interact with various applications executing on the additional user devices and to interact with the system 100. In certain embodiments, the additional user devices may be and/or may include a computer, a tattoo machine 120, a wireless battery pack 122, a hover device 124, a footswitch 126, any type of sensor, a laptop, a set-top-box, a tablet device, a phablet, a server, a mobile device, a smartphone, a smart watch, and/or any other type of computing device, and/or any combination thereof.

The first user device 102 and/or additional user devices may belong to and/or form a communications network. In certain embodiments, the communications network may be a local, mesh, or other network that enables and/or facilitates various aspects of the functionality of the system 100. In certain embodiments, the communications network may be formed between the first user device 102 and additional user devices through the use of any type of wireless or other protocol and/or technology. For example, user devices may communicate with one another in the communications network by utilizing any protocol and/or wireless technology, satellite, fiber, or any combination thereof. As another example, the first user device 102 and/or additional user devices may connect and communicate with each other by utilizing infrared radiation, Bluetooth connectivity, ZigBee, Z-Wave, any type of wireless protocol, radio technologies (e.g. radio frequency technologies or other radio technologies), or any combination thereof. For example, the devices in the system may couple and communicate with each other by utilizing radio frequency chips. In certain embodiments, the first user device 102 and/or additional user devices may include a wireless transceiver, module, and/or chip, such as a Bluetooth chip, near-field communications chip, or other similar technology to enable the second user device 106 to communicate with other devices in the system 100. Notably, the communications network may be configured to communicatively link with and/or communicate with any other network of the system 100 and/or outside the system 100.

In certain embodiments, the first user device 102 and additional user devices belonging to the communications network may share and exchange data with each other via the communications network. For example, the user devices may share information relating to the various components of the user devices, information associated with usage of the devices, information identifying the locations of the devices, information indicating the types of sensors that are contained in and/or on the devices, information identifying the applications being utilized on the devices, information identifying how the user devices are being utilized by a user, information identifying user profiles for users of the user devices, information identifying device profiles for the user devices, information identifying the number of devices in the communications network, information identifying devices being added to or removed from the communications network, any other information, or any combination thereof.

In addition to the first user 101, the system 100 may also include a second user 110. The second user 110 may be an individual seeking to obtain a tattoo, a user of the system 100, any type of user, or a combination thereof. The second user device 111 may be utilized by first user 101 (or potentially the second user 110) to transmit signals to request various types of content, services, and data provided by and/or accessible by communications network 135 or any other network in the system 100. In further embodiments, the second user 110 may be a robot, a computer, a humanoid, an animal, any type of user, or any combination thereof. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. screen, monitor, graphical user interface, etc.) that may enable the first user 101 to interact with various applications executing on the second user device 111 and to interact with the system 100. In certain embodiments, the second user device 111 may be a tattoo machine, a tattoo machine accessory, a computer, a laptop, a set-top-box, a tablet device, a phablet, a server, a mobile device, a smartphone, a smart watch, and/or any other type of computing device. Illustratively, the second user device 111 is shown as a computing device in FIG. 1 and the second user 110 is shown as an individual seeking to obtain a tattoo on one or more portions of the second user's 110 body.

In certain embodiments, the first user device 102, the additional user devices, and/or the second user device 111 may have any number of software applications and/or application services stored and/or accessible thereon. For example, the first user device 102, the additional user devices, and/or the second user device 111 may include applications for controlling the operative features and functionality of the system 100, applications for controlling any device of the system 100, interactive social media applications, biometric applications, cloud-based applications, VoIP applications, other types of phone-based applications, product-ordering applications, business applications, e-commerce applications, media streaming applications, content-based applications, media-editing applications, database applications, gaming applications, internet-based applications, browser applications, mobile applications, service-based applications, productivity applications, video applications, music applications, social media applications, any other type of applications, any types of application services, or a combination thereof. In certain embodiments, the software applications may support the functionality provided by the system 100 and methods described in the present disclosure. In certain embodiments, the software applications and services may include one or more graphical user interfaces so as to enable the first and/or second users 101, 110 to readily interact with the software applications. The software applications and services may also be utilized by the first and/or second users 101, 110 to interact with any device in the system 100, any network in the system 100, or any combination thereof. In certain embodiments, the first user device 102, the additional user devices, and/or the second user device 111 may include associated telephone numbers, device identities, or any other identifiers to uniquely identify the first user device 102, the additional user devices, and/or the second user device 111.

The system 100 may include a tattoo machine 120, which may be utilized by a tattoo artist to artfully place tattoos onto the skin of a user, such as a client. In certain embodiments, the tattoo machine 120 may include any number of components including, but not limited to, processors, memories, wireless chips, wireless modules, transceivers, displays (e.g. for displaying information associated with the operation of the tattoo machine 120), light emitting diodes (e.g. to show status information associated with the operation of the tattoo machine 120), power supplies, needles, needle cartridges (within which a needle may be secured), ports (e.g. USB or other data and/or power ports), tattoo grips, any other components, or a combination thereof. In certain embodiments, any number of devices may be connected wirelessly to the tattoo machine 120 and/or via the ports. In certain embodiments, data may be uploaded into the tattoo machine 120 and/or downloaded from the tattoo machine 120. In certain embodiments, the tattoo machine 120 may be configured in a modular fashion such that any desired combination of components may be attached to the tattoo machine 120 depending on the tattoo artist' preferences. In certain embodiments, the tattoo machine 120 may be configured to wirelessly communicate with any devices within range of the tattoo machine 120, such as, but not limited to, the first user device 102, the second user device 111, the hover device 124, the wireless battery pack 122, the footswitch 126, any other device, or a combination thereof. In certain embodiments, the tattoo machine 120 may include any number of controls to control the amount of voltage for the tattoo machine 120 (e.g. for adjusting needle run speed), control positioning of a grip of the tattoo machine 120, control power levels for the tattoo machine 120, control any aspect of the tattoo machine 120, or a combination thereof. Illustratively, the tattoo machine 120 generally has pen-style shape, however, in certain embodiments, the tattoo machine 120 may have any desired shape. In certain embodiments, the tattoo machine 120 may include a needle cartridge, a battery pack, and/or any other components.

The system 100 may also include a wireless battery pack 122, which may be utilized to deliver power to a tattoo machine 120 and/or other suitable device. The wireless battery pack 122 may also be utilized to control the tattoo machine 120 and/or any other device communicatively linked to the wireless battery pack 122. In certain embodiments, the wireless battery pack 122 may include any number of components including, but not limited to, processors, memories, wireless chips, wireless modules, transceivers, displays (e.g. for displaying information associated with the operation of the tattoo machine 120 and/or the wireless battery pack 122), light emitting diodes (e.g. to show status information associated with the operation of the tattoo machine 120 and/or wireless battery pack 122), sensors (e.g. motion sensors, pressure sensors, gesture-based sensors, temperature sensors, voltage sensors, any other sensors, or a combination thereof), any other components, or a combination thereof. In certain embodiments, the wireless battery pack 122 may be attachable to the body (e.g. such as via a receptacle) of the tattoo machine 120 so as to provide power to the tattoo machine 120. When the wireless battery pack 122 is detached from the body of the tattoo machine 120, the wireless battery pack 122 may stop delivering power to the tattoo machine 120. In certain embodiments, any number of devices may be connected wirelessly to the wireless battery pack 122 and/or via the ports. In certain embodiments, data may be uploaded into the wireless battery pack 122 and/or downloaded from the wireless battery pack 122. In certain embodiments, the wireless battery pack 122 may be configured in a modular fashion such that any desired combination of components may be attached to the wireless battery pack 122 depending on the tattoo artist' preferences. In certain embodiments, the wireless battery pack 122 may be configured to wirelessly communicate with any devices within range of the wireless battery pack 122, such as, but not limited to, the first user device 102, the second user device 111, the hover device 124, the tattoo machine 120, the footswitch 126, any other device, or a combination thereof. In certain embodiments, the wireless battery pack 122 may include any number of controls to control the amount of voltage for an attached tattoo machine 120 (e.g. for adjusting needle run speed), control power levels for the tattoo machine 120, control electronic give settings, control any aspect of the tattoo machine 120 and/or wireless battery pack 122, or a combination thereof. In certain embodiments, the wireless battery pack 122 may be configured to output detailed current battery level information, power status information, voltage information, any other information, or a combination thereof. In certain embodiments, any data may be transmitted back and forth between the wireless battery pack 122 and any device communicatively linked to the wireless battery pack 122.

The system 100 may also include a hover device 124, which may be utilized to deliver power to a tattoo machine 120 and/or other suitable device. The hover device 124 may also be utilized to control the tattoo machine 120 and/or any other device communicatively linked to the hover device 124. In certain embodiments, the hover device 124 may include any number of components including, but not limited to, processors, memories, wireless chips, wireless modules, transceivers, displays (e.g. for displaying information associated with the operation of the tattoo machine 120 and/or hover device 124), light emitting diodes (e.g. to show status information associated with the operation of the tattoo machine 120 and/or hover device 124), sensors (e.g. motion sensors, pressure sensors, gesture-based sensors, temperature sensors, voltage sensors, any other sensors, or a combination thereof), any other components, or a combination thereof. In certain embodiments, the hover device 124 may be attachable to the tattoo machine 120 (such as via a wire) or other accessory so as to provide power to the tattoo machine 120 and/or other accessory. When the hover device 124 is detached from the tattoo machine 120 or other accessory, the hover device 124 may stop delivering power to the tattoo machine 120 and/or other accessory. In certain embodiments, any number of devices may be connected wirelessly to the hover device 124 and/or via the ports. In certain embodiments, data may be uploaded into the hover device 124 and/or downloaded from the hover device 124. In certain embodiments, the hover device 124 may be configured in a modular fashion such that any desired combination of components may be attached to the hover device 124 depending on the tattoo artist' preferences. In certain embodiments, the hover device 124 may be configured to wirelessly communicate with any devices within range of the hover device 124, such as, but not limited to, the first user device 102, the second user device 111, the wireless battery pack 122, the tattoo machine 120, the footswitch 126, any other device, or a combination thereof. In certain embodiments, the hover device 124 may include any number of controls to control the amount of voltage for an attached tattoo machine 120 (e.g. for adjusting needle run speed) or other accessory, control power levels for the tattoo machine 120, control electronic give settings, control any aspect of the tattoo machine 120 and/or other attached accessory, or a combination thereof. In certain embodiments, the hover device 124 may be configured to output detailed current battery level information, power status information, voltage information, any other information, or a combination thereof. In certain embodiments, the hover device 124 may include a timer so that a tattoo artist can time a tattoo session. In certain embodiments, the hover device 124 may include any number of motion/gesture-based sensors that can detect the motion of a user's fingers and/or hands within range of the sensors to control the operative functions of the hover device 124. For example, placing the user's hand over the entirety of hover device 124 may cause the hover device 124 to activate or deactivate. As another example, placing a finger over one side of the hover device 124 may cause an increase in voltage and a finger over the other side of the hover device 124 may cause a decrease in voltage. Similarly, any other controls of the hover device 124 may be controlled by any number of gestures and/or movements.

The system 100 may also include a footswitch 126, which may be utilized to control any device communicatively coupled to the footswitch 126. A user may use the user's foot and/or toes to contact various portions, buttons, and/or input interfaces of the footswitch 126 to control a device connected to the footswitch 126. For example, the footswitch 126 may be configured to adjust the voltage of a connected tattoo machine, power levels, electronic give settings, transfer of data between connected devices, any other controls described herein, or a combination thereof. In certain embodiments, footswitch 126 may include any number of components including, but not limited to, processors, memories, wireless chips, wireless modules, transceivers, displays (e.g. for displaying information associated with the operation of the tattoo machine 120 and/or footswitch 126), light emitting diodes (e.g. to show status information associated with the operation of the tattoo machine 120 and/or footswitch 126), sensors (e.g. motion sensors, pressure sensors, gesture-based sensors, temperature sensors, voltage sensors, any other sensors, or a combination thereof), any other components, or a combination thereof. In certain embodiments, the footswitch 126 may be communicatively linked to a tattoo machine 120 and/or other accessory via wireless communication. In certain embodiments, any number of devices may be connected wirelessly to the footswitch 126 and/or via the ports. In certain embodiments, data may be uploaded into the footswitch 126 and/or downloaded from the footswitch 126. In certain embodiments, the footswitch 126 may be configured in a modular fashion such that any desired combination of components may be attached to the footswitch 126 depending on the tattoo artist' preferences. In certain embodiments, the footswitch 126 may be configured to wirelessly communicate with any devices within range of the footswitch 126, such as, but not limited to, the first user device 102, the second user device 111, the wireless battery pack 122, the tattoo machine 120, the the hover device 124, any other device, or a combination thereof.

The system 100 may also include a communications network 135. The communications network 135 may be under the control of a service provider, the first user 101, the second user 110, any other designated user, a computer, another network, or a combination thereof. The communications network 135 of the system 100 may be configured to link each of the devices in the system 100 to one another. For example, the communications network 135 may be utilized by the first user device 102 to connect with other devices within or outside communications network 135, such as, but not limited to, the tattoo machine 120, the wireless battery pack 122, the hover device 124, the footswitch 126, and/or any other device. Additionally, the communications network 135 may be configured to transmit, generate, and receive any information and data traversing the system 100. In certain embodiments, the communications network 135 may include any number of servers, databases, or other componentry. The communications network 135 may also include and be connected to a mesh network, a local network, a cloud-computing network, an IMS network, a VoIP network, a security network, a VoLTE network, a wireless network, an Ethernet network, a satellite network, a broadband network, a cellular network, a private network, a cable network, the Internet, an internet protocol network, MPLS network, a content distribution network, any network, or any combination thereof. Illustratively, servers 140, 145, and 150 are shown as being included within communications network 135. In certain embodiments, the communications network 135 may be part of a single autonomous system that is located in a particular geographic region, or be part of multiple autonomous systems that span several geographic regions.

Notably, the functionality of the system 100 may be supported and executed by using any combination of the servers 140, 145, 150, and 160. The servers 140, 145, and 150 may reside in communications network 135, however, in certain embodiments, the servers 140, 145, 150 may reside outside communications network 135. The servers 140, 145, and 150 may provide and serve as a server service that performs the various operations and functions provided by the system 100. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 145 may include a memory 146 that includes instructions, and a processor 147 that executes the instructions from the memory 146 to perform the various operations that are performed by the server 145. Furthermore, the server 150 may include a memory 151 that includes instructions, and a processor 152 that executes the instructions from the memory 151 to perform the various operations that are performed by the server 150. In certain embodiments, the servers 140, 145, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 145, 150 may be communicatively linked to the communications network 135, any network, any device in the system 100, or any combination thereof.

The database 155 of the system 100 may be utilized to store and relay information that traverses the system 100, cache content that traverses the system 100, store data about each of the devices in the system 100 and perform any other typical functions of a database. In certain embodiments, the database 155 may be connected to or reside within the communications network 135, any other network, or a combination thereof. In certain embodiments, the database 155 may serve as a central repository for any information associated with any of the devices and information associated with the system 100. Furthermore, the database 155 may include a processor and memory or be connected to a processor and memory to perform the various operation associated with the database 155. In certain embodiments, the database 155 may be connected to the servers 140, 145, 150, 160, the first user device 102, the second user device 111, the additional user devices, any devices in the system 100, any process of the system 100, any program of the system 100, any other device, any network, or any combination thereof.

The database 155 may also store information and metadata obtained from the system 100, store metadata and other information associated with the first and second users 101, 110, store sensor data from sensors of the tattoo machine 120 and/or tattoo machine accessories, store voltage setting data, store e-give setting data, store power status and/or power level information, store information relating to failures of components of the tattoo machine 120 and/or tattoo machine accessories, store user profiles associated with the first and second users 101, 110, store device profiles associated with any device in the system 100, store communications traversing the system 100, store user preferences, store information associated with any device or signal in the system 100, store information relating to patterns of usage relating to the user devices 102, 111 and/or additional user devices, store any information obtained from any of the networks in the system 100, store historical data associated with the first and second users 101, 110, store device characteristics, store information relating to any devices associated with the first and second users 101, 110, store information associated with the communications network 135, store any information generated and/or processed by the system 100, store any of the information disclosed for any of the operations and functions disclosed for the system 100 herewith, store any information traversing the system 100, or any combination thereof. Furthermore, the database 155 may be configured to process queries sent to it by any device in the system 100.

Operatively, the system 100 may operate and/or execute the functionality as described in the methods (e.g. method 2900 as described below) of the present disclosure and in the following description. Referring now also to FIGS. 2-26, a process flow for using the program/application providing the operative functionality of the system 100 is shown. For example, the process flow may be utilized create an account with an application, scan for devices within range of a device running the application, pair detected devices, and/or control the paired devices. To that end, in FIG. 2, an interface screen 200 is shown, which enables a user, such as first user 101, to view the opening interface of the application supporting the functionality of the system 100. The user may be allowed to skip this opening screen by selecting a link rendered on the interface screen 200. When the user skins the opening screen, the user may be taken to interface screen 300, which may enable a user to login to an existing account with the application, and/or register and create a new account with the application. If the user has an existing account, the user may authenticate into the account by using a password-username combination, multi-factor authentication, and/or biometric authentication (e.g. face scan, fingerprint scan, etc.) If the user does not have an existing account, the user may selection the option to create an account and may be taken to interface screen 400, which may request the user to input a username, password, email address, birth date, demographic information, personal preference information, any other information, or a combination thereof. Once the user inputs the required user account information and agrees to the terms of use, the account may be created for the user.

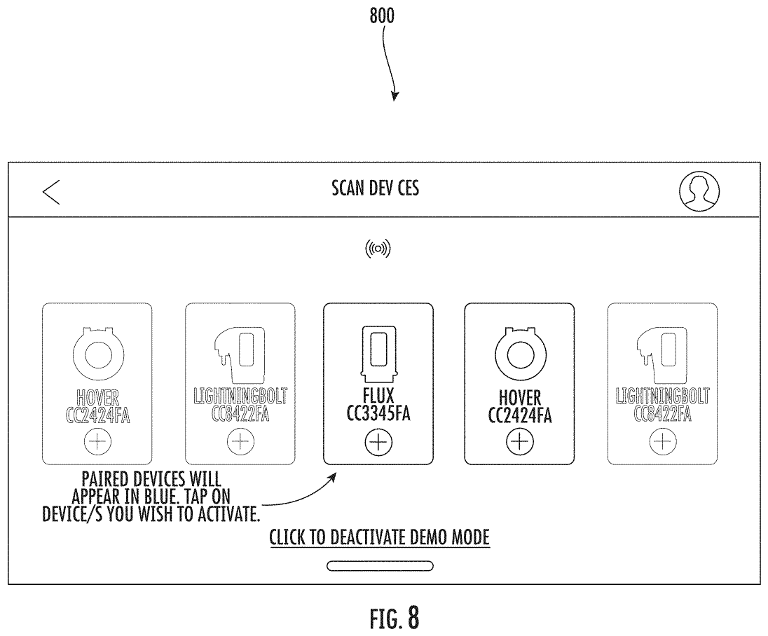

In the event the user forgets his password or is otherwise unable to authenticate into the user's account, user interface screen 500 may be shown. User interface screen 500 may enable a user to retrieve his account by entering in an email address associated with the account, a phone number associated with the account, and/or other information associated with the account. Username and/or password information may be transmitted to the user's email address and/or phone so that the user may retrieve the user's account. Once the user has ultimately logged into the user's account, the user may be presented with a plurality of options for interacting with the application. For example, user interface screen 600 may be displayed to the user, which may enable the user to select a link to scan for devices within communication range of the first user device 102 (or other device) executing the application. When the option is selected, the first user device 102 may scan for devices within the communication range of the first user device. For example, as shown in interface screen 700, the first user device 102 may detect that a tattoo machine 120, wireless battery packs 122, hover devices 124 are within range of the communication device. A visual identification confirming the detection of devices may be shown on the interface screen 700. The interface screen 700 may give the user the option to pair the first user device 102 and/or application with any of the detected devices. For example, in interface screen 800, the user may have option to pair the tattoo machine 120 with the hover device 124 and/or with the application and the first user device 102. A visual indication may be displayed confirming that an effective pairing/synchronization has occurred, as shown in FIG. 8.

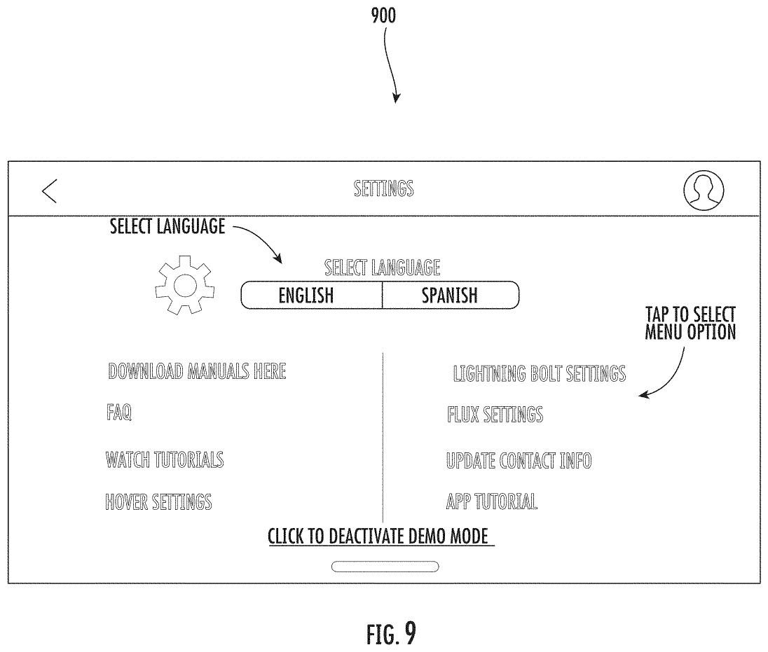

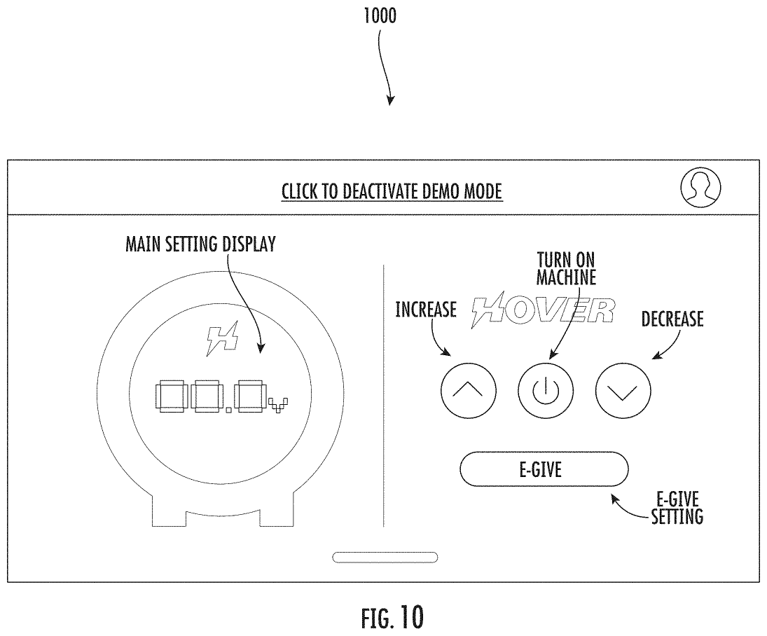

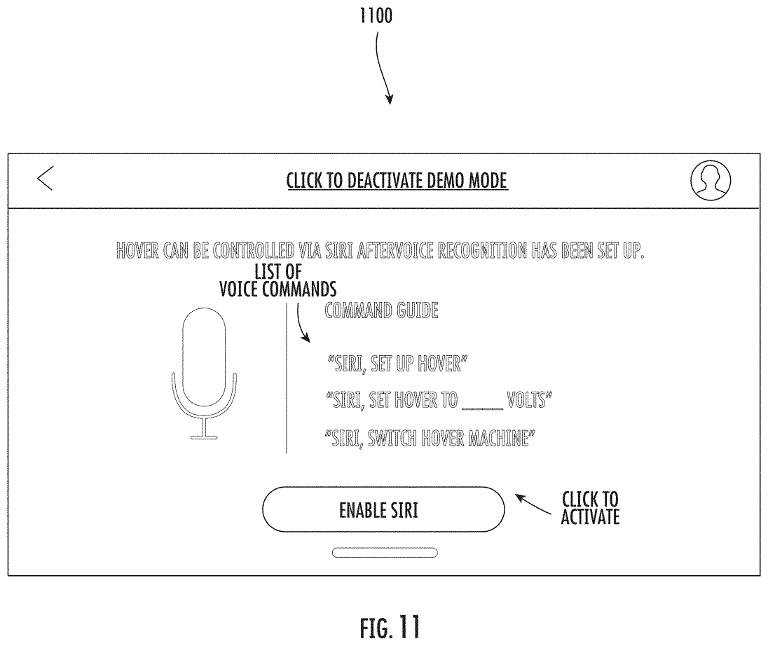

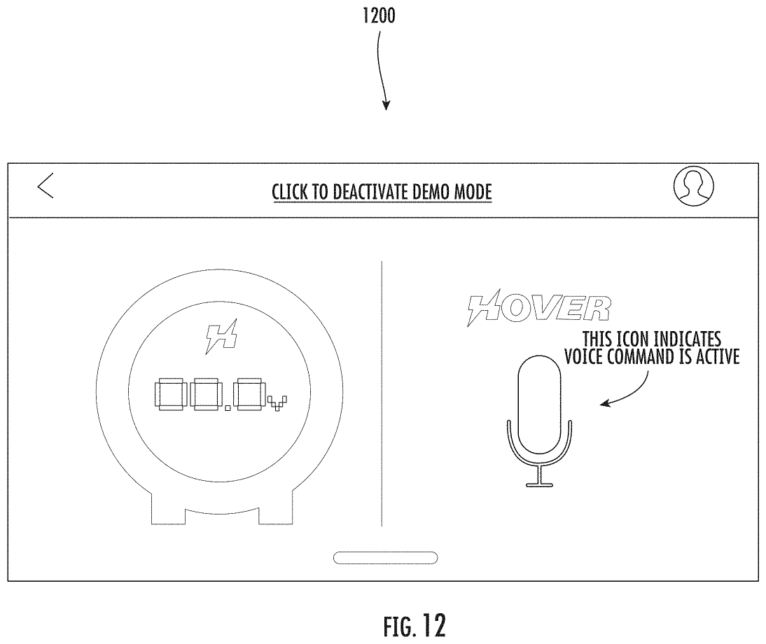

Interface screen 900 displays a sample settings feature of the application. The settings may enable the user to set the desired language for the application, download user manuals for devices, access a frequently asked questions section, watching tutorials for using devices, access and change settings for each of the detected devices (e.g. voltage settings, current settings, power level settings, mode of operation settings, configuration settings, any other settings, or a combination thereof), access a tutorial for using the application, among other desired settings. In FIG. 9, the user may opt to select English as the preferred language for the application. User interface screen 1000 of FIG. 10 provides an example of when the user selects the option to access hover device 124 settings. The settings may enable the user to activate or deactivate the hover device 124, increase or decrease the voltage, and adjust electronic give settings. In certain embodiments, a graphical representation of the hover device 124 may be rendered in the interface screen 1000 to show the current settings of the hover device 124 in real time. FIG. 11 provides an interface screen 1100 for enabling a user to control paired devices through the user of any number of voice commands. In certain embodiments, the voice commands may be preset commands, such as to set up the hover device 124, change voltage settings, or switch from one hover machine 124 to another hover machine 124. In certain embodiments, the application may enable the user to create their own personalized voice commands to control any aspect of a paired device. In certain embodiments, the application may enable the application to communicate with a voice command program, such as SIRI, which may be utilized to receive and process the voice commands for the application. User interface screen 1200 of FIG. 12 may be displayed when the user opts to utilize voice commands. For example, in screen 1200 a visual identification is rendered to show that voice commands have been activated for controlling the hover device 124 (or other paired device).

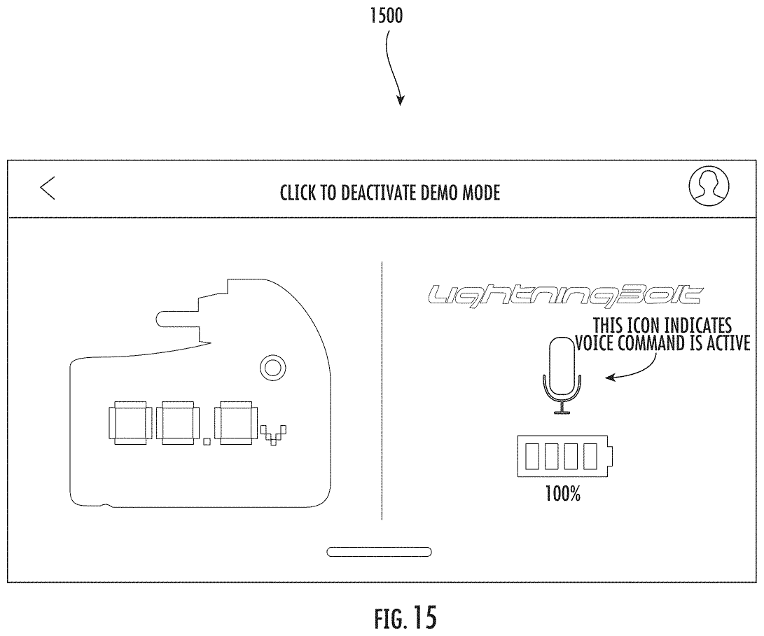

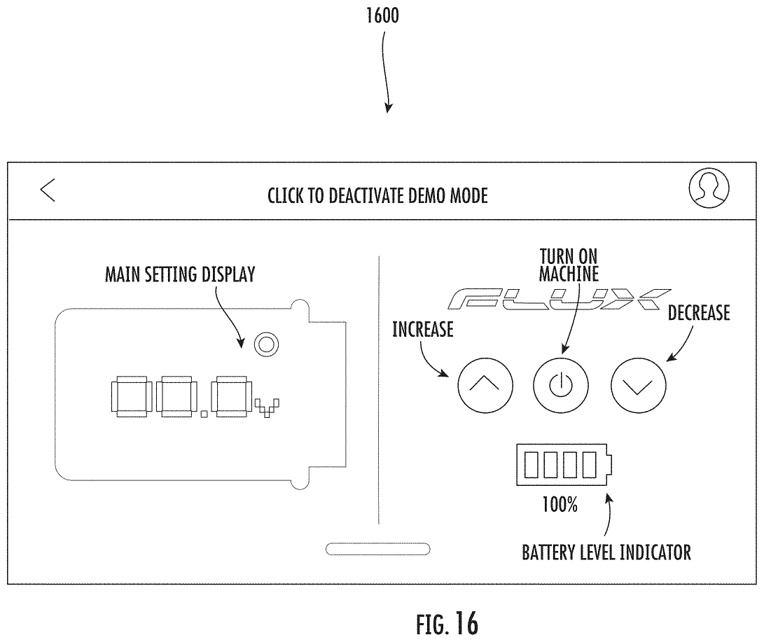

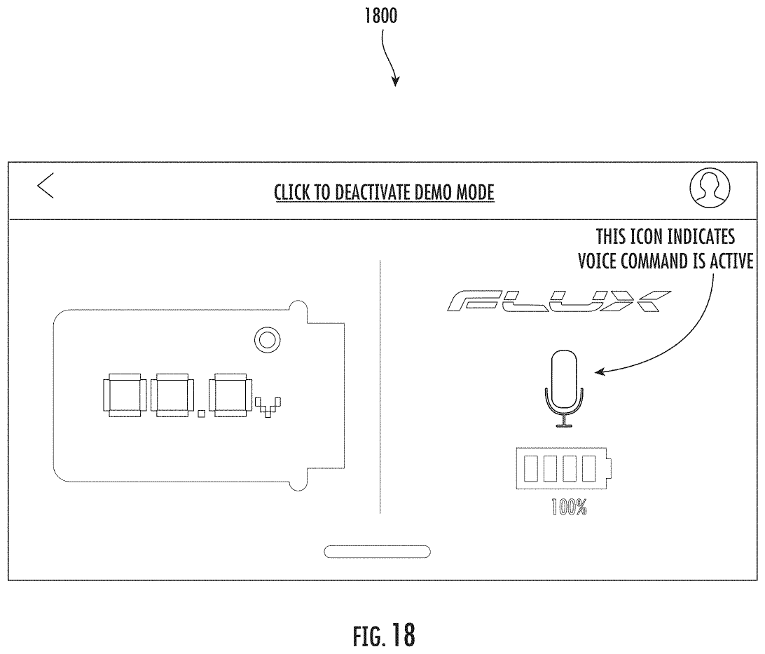

User interface screen 1300 illustrates the ability to set settings for another device, in this case a paired wireless battery pack 122. In certain embodiments, the user interface screen 1300 may enable the user to view a graphical representation of the current status of the wireless battery pack 122, increase or a decrease voltage, activate or deactivate the wireless battery pack 122 and/or a device connected to the wireless battery pack 122, a battery level indicator that indicates how much power is left in the wireless battery pack 122, electronic give settings, any other settings, or a combination thereof. As with the hover device 124, user interface screen 1400 may enable the user to activate voice commands to control the wireless battery pack 122. User interface screen 1500 of FIG. 15 illustrates a visual confirmation indicating that the voice commands for controlling the wireless battery pack 122 have been activated by the user via the application. User interface screen 1600 of FIG. 16 illustrates the ability to set settings for yet another device, in this case a paired tattoo machine 120 (e.g. flux machine). in certain embodiments, the user interface screen 1600 may enable the user to view a graphical representation of the current status of the tattoo machine 120 or a portion of the tattoo machine 120, increase or decrease voltage, activate or deactivate the tattoo machine 120, a battery level indicator, change electronic give settings for using the tattoo machine 120, any other settings, or a combination thereof. As with the hover device 124 and the wireless battery pack 122, user interface screen 1700 may enable the user to activate voice commands to control the tattoo machine 120. User interface screen 1800 of FIG. 18 illustrates a visual confirmation indicating that the voice commands for controlling the tattoo machine 120 have been activated by the user via the application.

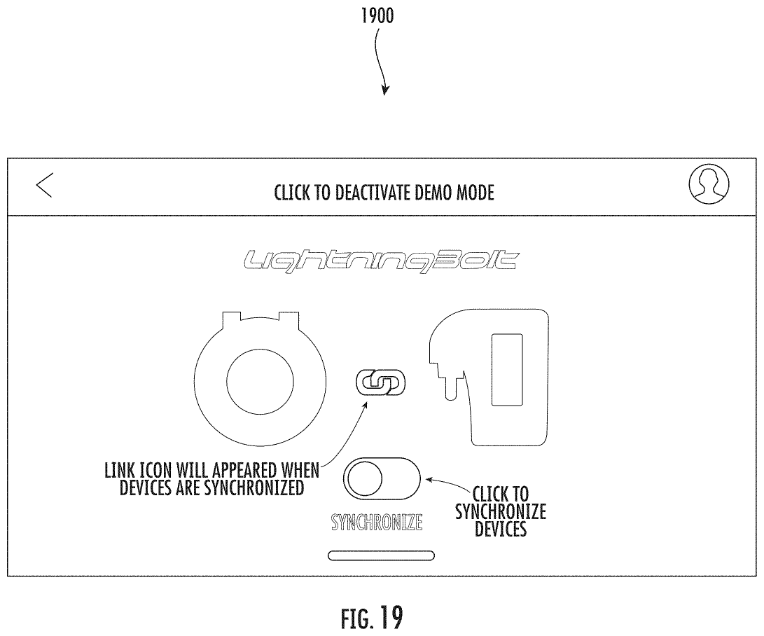

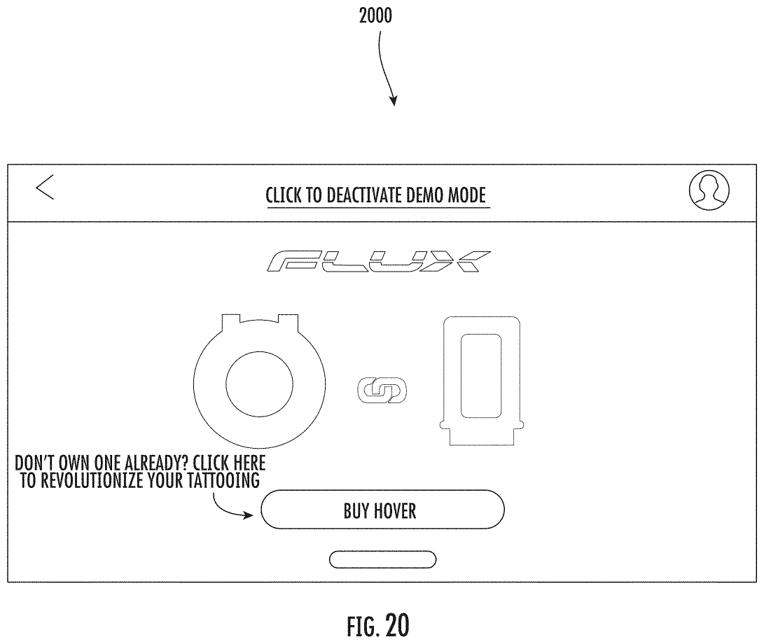

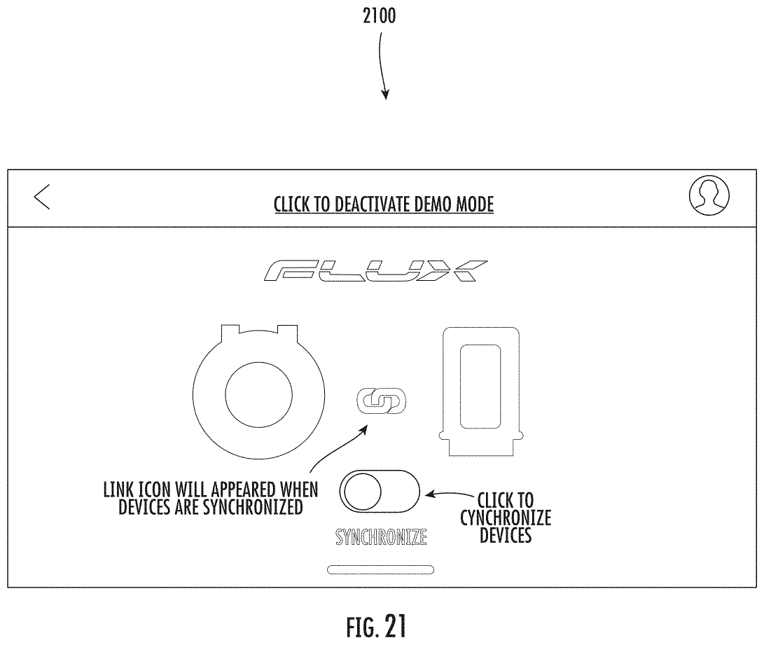

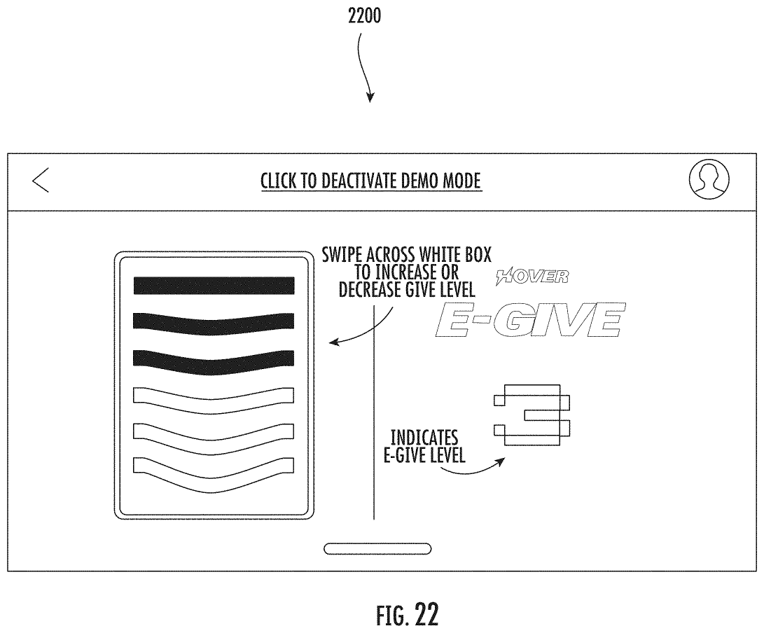

User interface screen 1900 of FIG. 19 provides the option to pair/synchronize detected devices with each other. For example, interface screen 1900 shows that the user decided to pair/synchronize a hover device 124 with a wireless battery pack 122. During the pairing/synchronization process, the devices may be communicatively coupled with each other. Additionally, the devices may share information back and forth about each connected device. In certain embodiments, commands from one device may be utilized to control any aspect of a paired device, such as is described in the present disclosure. User interface screen 2000 of FIG. 20 illustrates the capability of the application to enable the user to buy any item, such as the hover device 124, directly using the application. In certain embodiments, the application may recommend certain devices or products to buy based on the user's preferences as indicated in the user's profile, the user's use of the application itself, and/or the devices that the user utilizes, such as during a tattoo session. User interface screen 2100 of FIG. 21 illustrates the capability provided by the application to pair another set of devices together. In this case, interface screen 2100 illustrates that the user decided to pair/synchronize a hover device 124 with a tattoo machine 120 (or a portion of a tattoo machine, such as a battery pack of the tattoo machine 120). User interface screen 2200 of FIG. 12 illustrates the capability for the user to adjust electronic give settings for a paired device. For example, the user may swipe across (or up or down) the visually rendered box to increase or decrease the give level. Additionally, a numeric rendering of the current give level may also be displayed, as shown in FIG. 22. In certain embodiments, the application and/or devices may calculate, on a particular power supply (e.g. hover device 124, wireless battery pack 122, portion of tattoo machine 120, etc.), a targeted voltage drop related to pressure of the tattoo needle of the tattoo machine 120 against a user's skin, which may correspond to the electronic give. In certain embodiments, a give response for a machine (e.g. tattoo machine 120) can also be varied by a plurality of give levels, with each give level having its own threshold and rate of change to the observed current exceeding or falling below a threshold current. In this way, a range of give emulations are possible, ranging from “hard” give to “soft” give, which correspond to the give levels that may be set via the application. In some embodiments, the give level may range from zero to seven, with zero representing no voltage adjustment (i.e. normal operation of the machine without give adjustment), one emulating a “hard” give, seven emulating a “soft” give, and the levels between one and seven ranging from the hardest give to the softest give. Certain embodiments may include greater or fewer give levels. For example, the give level range may range from zero to five or to any number. In certain embodiments, the give level may range from a number other than zero to any other desired number. In one embodiment, the give level may be binary (on or off). In certain embodiments, the give levels may range from zero to about twenty. In certain embodiments, the give level may be utilized to provide control over the speed of, or power delivered to, a needle and impact force of the needle into a user's skin.

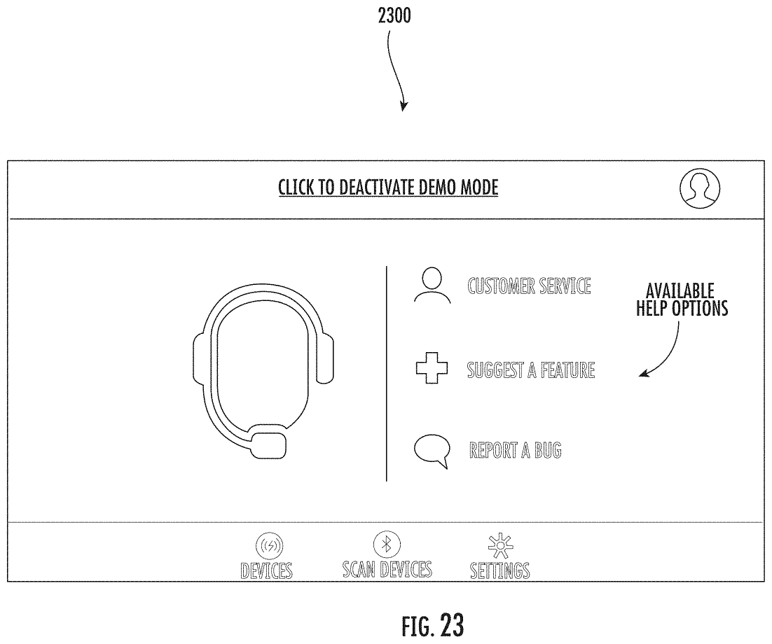

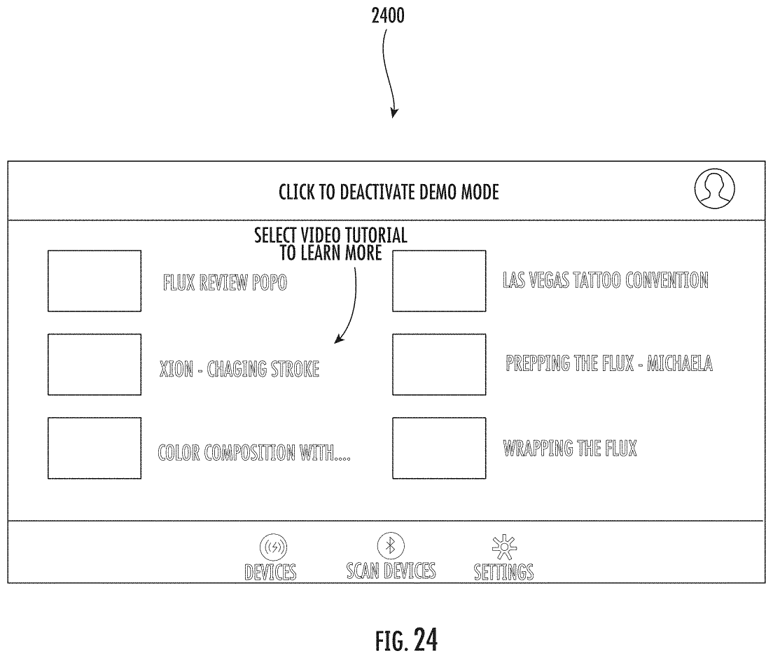

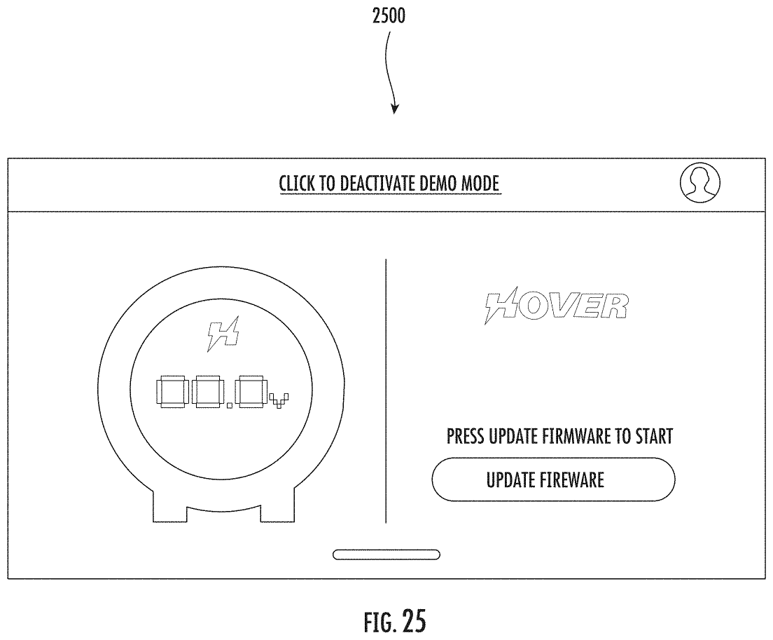

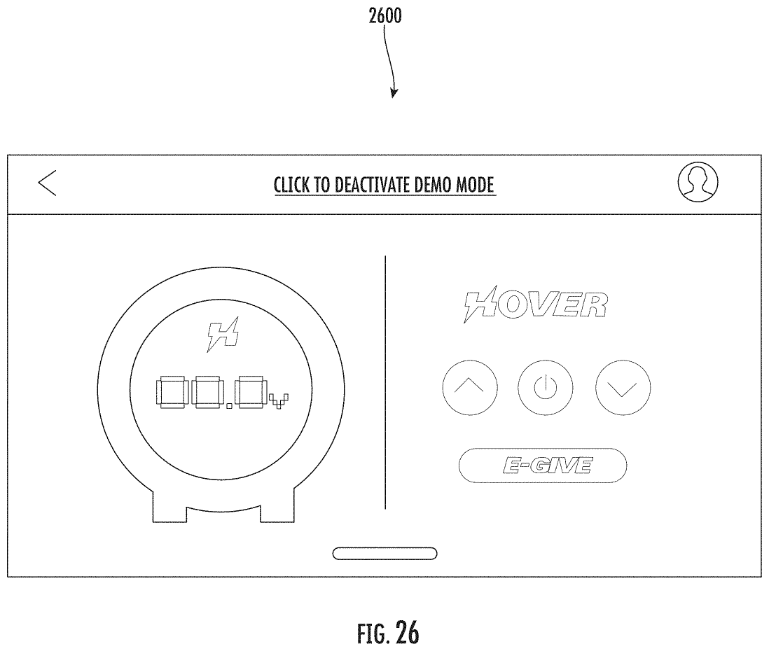

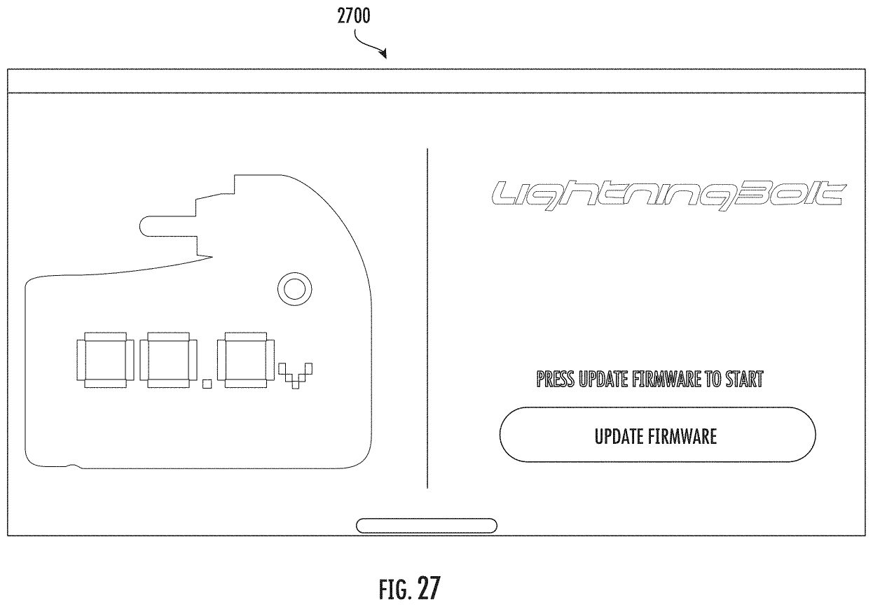

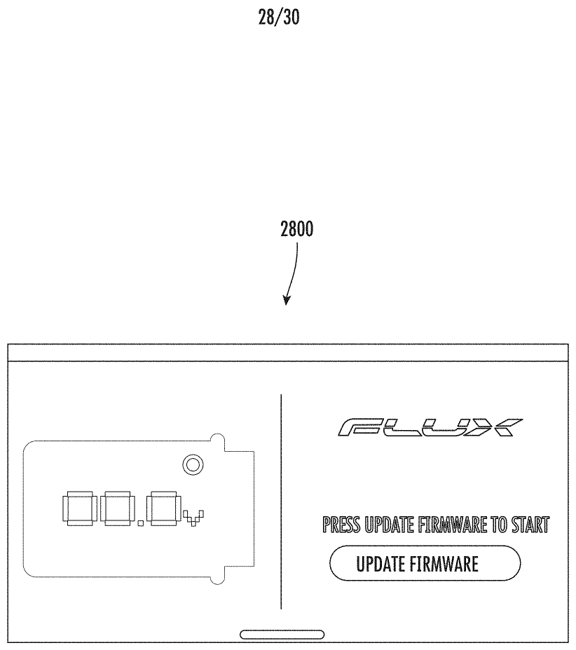

User interface screen 2300 of FIG. 23 illustrates the ability of the application to enable the user to contact customer service for assistance, suggest a feature to be added to the application, and/or report a failure or bug associated with the application and/or any paired devices. User interface screen 2400 of FIG. 24 illustrates sample video tutorials that may be hosted in the application, which may instruct a user to utilize the various devices, create a tattoo, sterilize equipment, or a host of other topics of interest. User interface screen 2500 of FIG. 25 illustrates the capability of the application to allow a user to update the firmware of any paired device. Illustratively, interface screen 2500 depicts the option to update the firmware for the hover device 124. User interface screen 2600 of FIG. 26 may be a sample rendering of a screen of the application through which the user can adjust settings of the hover device 124. Similar screens may be utilized to display settings for other paired devices as well. For example, user interface screen 2700 of FIG. 27 illustrates the capability to update the firmware for a wireless battery pack 122 (e.g. LightningBolt machine), such as via a digital link or button presented on the user interface screen 2700. User interface screen 2800 of FIG. 28 may be a sample rendering of a screen of the application that allows a user to update the firmware of a tattoo machine 120 (e.g. flux machine). In certain embodiments, if a user opts to update the firmware of a particular device, the user interface may display an update progress bar or other indicator that indicates to the user the progress of the update in real-time.

Notably, as shown in FIG. 1, the system 100 may perform any of the operative functions disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, facilitating authentication of a user of a user account for use with an application controlling the tattoo machine 120 and/or tattoo machine accessories; scanning for devices within a communication range of a user device; detecting devices within communication range of the user device; providing an option to pair one or more of the detected devices with the user device and/or application; pairing the devices according to user preferences; enabling control of the paired devices; adjusting settings of the paired devices; unpairing paired devices; tracking data associated with operation of the paired devices; and performing any other suitable operations conducted in the system 100 or otherwise. In one embodiment, multiple servers 160 may be utilized to process the functions of the system 100. The server 160 and other devices in the system 100, may utilize the database 155 for storing data about the devices in the system 100 or any other information that is associated with the system 100. In one embodiment, multiple databases 155 may be utilized to store data in the system 100.

Although FIGS. 1-30 illustrates specific example configurations of the various components of the system 100, the system 100 may include any configuration of the components, which may include using a greater or lesser number of the components. For example, the system 100 is illustratively shown as including a first user device 102, a second user device 111, a tattoo machine 120, a wireless battery pack 122, a hover device 124, a footswitch 126, a communications network 135, a server 140, a server 145, a server 150, a server 160, and a database 155. However, the system 100 may include multiple first user devices 102, multiple second user devices 111, multiple tattoo machines 120, multiple wireless battery packs 122, multiple hover devices 124, multiple footswitches 126, multiple communications networks 135, multiple servers 140, multiple servers 145, multiple servers 150, multiple servers 160, multiple databases 155, or any number of any of the other components inside or outside the system 100. Furthermore, in certain embodiments, substantial portions of the functionality and operations of the system 100 may be performed by other networks and systems that may be connected to system 100.

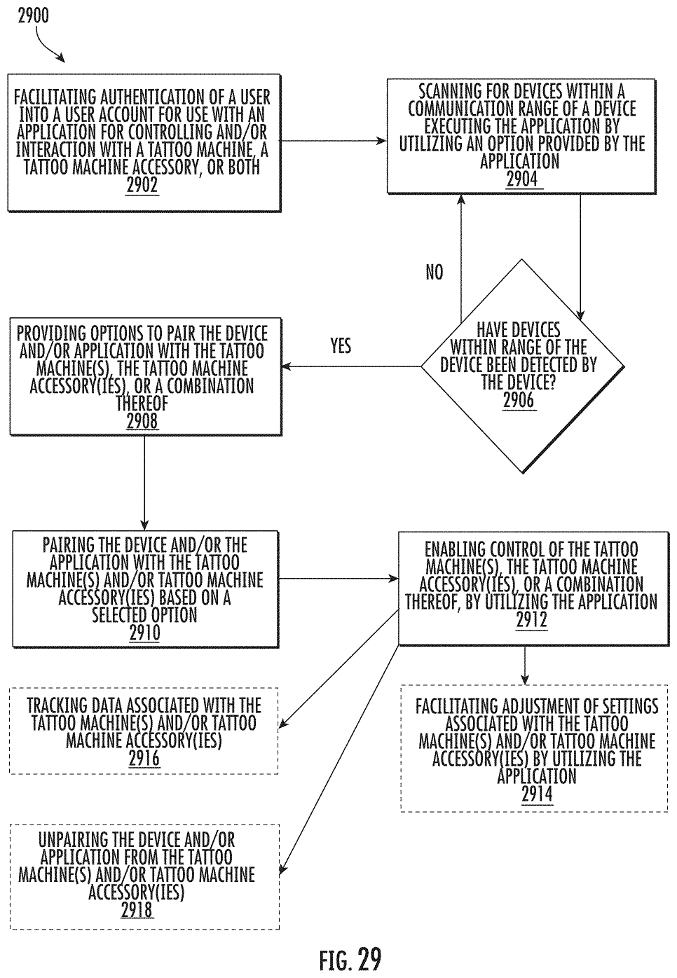

Notably, the system 100 may execute and/or conduct the functionality as described in the method(s) that follow. As shown in FIG. 29, an exemplary method 2900 for control of tattoo machines and/or tattoo machine accessories is schematically illustrated. The method 2900 and/or functionality and features supporting the method 2900 may be conducted via an application of the system 100, devices of the system 100, processes of the system 100, any component of the system 100, or a combination thereof. The method 2900 may include steps for authenticating a user (e.g. first user 101) into the application, scanning for devices to be paired using the application and/or a user device, pairing the detected devices, enabling control of the paired devices, adjusting settings of the paired devices, tracking data associated with usage of the devices, among other steps and functionality. To that end, the method 2900 may, at step 2902, include facilitating authentication of a user into a user account of an application that may be utilized to control tattoo machines, tattoo machine accessories, other devices, or a combination thereof. For example, the user may enter in a username and password combination, a biometric identifier (e.g. face scan, iris scan, fingerprint scan, etc.), and/or other authentication credentials into a user interface of an application executing on the first user device 102 (or other device of system 100) to log into the user account. The user account may store the user's information, demographic information, user preferences associated with using the tattoo machine 120 and/or tattoo machine accessories (e.g. tattoo machine settings, etc.), media content, other user preferences, tattoo preferences, previously completed tattoos, future tattoos to be completed, scheduled tattoo sessions, tattoo ink preferences, any other information, or a combination thereof. In certain embodiments, the authentication into the user account may be performed and/or facilitated by the first user 101, the second user 110 and/or by utilizing the first user device 102, the second user device 111, the server 140, the server 145, the server 150, the server 160, the communications network 135, any combination thereof, or by utilizing any other appropriate program, network, system, or device.

At step 2904, the method 2900 may include providing an option to scan for devices within a communication range of the first user device 102 utilized by the first user. In certain embodiments, at step 2904, the method 2900 may also include scanning for devices within a communication range of the first user device 102 (or other device that the first user 101 is using). For example, the application executing on the first user device 102 may cause a wireless chip (or module, transceiver, and/or other communication device) of the first user device 102 to scan for other devices in the communication range of the first user device 102. In certain embodiments, the scanning may be performed and/or facilitated by the first user 101, the second user 110 and/or by utilizing the first user device 102, the second user device 111, the tattoo machine 120, the wireless battery pack 122, the hover device 124, the footswitch 126, the server 140, the server 145, the server 150, the server 160, the communications network 135, any combination thereof, or by utilizing any other appropriate program, network, system, or device. At step 2906, the method 2900 may include determining whether any devices within communication range of the first user device 102 have been detected based on the scanning. In certain embodiments, the determining may be performed and/or facilitated by the first user 101, the second user 110 and/or by utilizing the first user device 102, the second user device 111, the tattoo machine 120, the wireless battery pack 122, the hover device 124, the footswitch 126, the server 140, the server 145, the server 150, the server 160, the communications network 135, any combination thereof, or by utilizing any other appropriate program, network, system, or device.

If, at step 2906, no devices have been detected to be within communication range of the first user device 102, the method 2900 may revert back to step 2904 and continue scanning for devices within the communication range of the first user device 102. In certain embodiments, if no devices have been detected after a selected period of time, the scanning may be ceased and the first user 101 may be provided with an option to restart scanning at another time. If, however, step 2906, devices have been detected to be within communication range of the first user device 102, the method 2900 may proceed to step 2908. At step 2908, the method 2900 may include providing an option(s) to pair the first user device 102 and/or the application executing on the first user device 102 with any one or more of the detected devices. For example, the user interface of the application may display an indication that indicates which devices have been detected and may provide an option to pair one or more of the detected devices with each other and/or with the first user device 102 and/or the application. By way of example only, the first user device 102 may detect that tattoo machine 120, wireless battery pack 122, and footswitch 126 are within communication range of the first user device 102. In certain embodiments, the providing of the option(s) may be performed and/or facilitated by the first user 101, the second user 110 and/or by utilizing the first user device 102, the second user device 111, the tattoo machine 120, the wireless battery pack 122, the hover device 124, the footswitch 126, the server 140, the server 145, the server 150, the server 160, the communications network 135, any combination thereof, or by utilizing any other appropriate program, network, system, or device.

At step 2910, the method 2900 may include pairing and/or synchronizing the first user device 102 and/or application and/or the detected devices with each other according to the option selected by the first user 101. As an example, the first user 101 may have selected the option to pair the first user device 102 and application with the tattoo machine 120 and the wireless battery pack 122, but not the footswitch 126. In such a scenario, the first user device 102 may utilize a wireless chip (or other communications means) of the first user device 102 to couple the first user device 102 wirelessly to the tattoo machine 120 and wireless battery pack 122. In certain embodiments, the pairing and/or synchronizing may be performed and/or facilitated by the first user 101, the second user 110 and/or by utilizing the first user device 102, the second user device 111, the tattoo machine 120, the wireless battery pack 122, the hover device 124, the footswitch 126, the server 140, the server 145, the server 150, the server 160, the communications network 135, any combination thereof, or by utilizing any other appropriate program, network, system, or device. At step 2912, the method 2900 may include enabling control of the paired devices, such as by utilizing the application. Using the example, above, once the first user device 102 is paired with the tattoo machine and the wireless battery pack 122, the first user 101 may control the tattoo machine 120 and/or the wireless battery pack 122 by utilizing the first user device 102 and/or the application. In certain embodiments, the enabling of the control of the paired devices may be performed and/or facilitated by the first user 101, the second user 110 and/or by utilizing the first user device 102, the second user device 111, the tattoo machine 120, the wireless battery pack 122, the hover device 124, the footswitch 126, the server 140, the server 145, the server 150, the server 160, the communications network 135, any combination thereof, or by utilizing any other appropriate program, network, system, or device.

At step 2914, which may be optional, the method 2900 may include, as an example of control, facilitating adjustment of settings associated with the paired devices. The first user 101 may adjust settings associated with the tattoo machine 120, the wireless battery pack 122, or both. Such settings may include, but are not limited to, voltage settings (e.g. increasing or decreasing the voltage for the tattoo machine 120 to adjust the needle run speed for a needle attached to the tattoo machine 120), electronic give settings (e.g. increasing or decreasing the give level), current settings (increasing or decreasing the amount of current delivered), configuration settings (e.g. causing the physical configuration of the tattoo machine 120 or wireless battery pack 122 to be adjusted), any other settings, or a combination thereof. In certain embodiments, the adjusting of settings may be performed and/or facilitated by the first user 101, the second user 110 and/or by utilizing the first user device 102, the second user device 111, the tattoo machine 120, the wireless battery pack 122, the hover device 124, the footswitch 126, the server 140, the server 145, the server 150, the server 160, the communications network 135, any combination thereof, or by utilizing any other appropriate program, network, system, or device. At step 2916, which may be optional, the method 2900 may include, as another example of control, tracking data associated with the paired devices. In the example above, the first user device 102, via the application, may track data associated with the tattoo machine 120 and/or the wireless battery pack 122. Such data may include, but is not limited to, data obtained via any of the sensors of the paired devices, voltage readings, current readings, needle velocity, motion readings, temperature readings, pressure readings, power level readings, failures associated with components of the paired devices, any other obtainable data, or a combination thereof. In certain embodiments, the tracking of the data may be performed and/or facilitated by the first user 101, the second user 110 and/or by utilizing the first user device 102, the second user device 111, the tattoo machine 120, the wireless battery pack 122, the hover device 124, the footswitch 126, the server 140, the server 145, the server 150, the server 160, the communications network 135, any combination thereof, or by utilizing any other appropriate program, network, system, or device.

At step 2918, which be optional, the method 2900 may include enabling the first user 101 (or first user device 102) to unpair one or more of the paired devices from each other and/or from the first user device 102 and/or application. In certain embodiments, the unpairing may be performed and/or facilitated by the first user 101, the second user 110 and/or by utilizing the first user device 102, the second user device 111, the tattoo machine 120, the wireless battery pack 122, the hover device 124, the footswitch 126, the server 140, the server 145, the server 150, the server 160, the communications network 135, any combination thereof, or by utilizing any other appropriate program, network, system, or device. In addition to the foregoing steps, the method 2900 may also incorporate additional functionality. For example, the method 2900 may enable the first user 101 and/or first user device 102, such as via the application, to book and schedule tattoo sessions with potential users, provide digital release authorizations to allow a user to consent to being tattooed, set tattoo session timers, photograph a tattoo (e.g. such as by using a camera of the first user device 102), upload media content corresponding to a tattoo to be tattooed on a user, detect component failures and/or problems of the paired devices, track whether supplies associated with the tattoo machines 120 and/or tattoo machine accessories need to be replenished, order replacement supplies, order tattoo machines 120 and/or tattoo machine accessories, scan an image or media content of interest (such as for inspiration for a tattoo), receive feedback from the user and/or other users relating to the tattooing process or the tattoo work itself, post content to social media or online platforms, and/or any other functionality as described in the present disclosure. Notably, the method 2900 may further incorporate any of the features and functionality described for the system 100, any other method disclosed herein, or as otherwise described herein.

The systems and methods disclosed herein may include additional functionality and features. In certain embodiments, the system 100 may enable the first user device 102 and/or application to pair with any type of tattoo machine accessory, such as a permanent makeup tattoo machine accessory. In certain embodiments, the application may enable a user to contact the first user 101 (or second user 110) via a communication portal of the application. The communication portal may enable direct messaging, email messaging, chat messaging, phone calls, any type of communications, or a combination thereof. In certain embodiments, the application may enable customers to place orders for tattoo equipment and/or supplies directly via the application and/or via third party sites linked to the application. In certain embodiments, the system 100 may enable an artist and/or tattoo studio to upload a digital release into the application and have a tattoo client electronical sign the release so that the tattoo session may begin. Additionally, the digitally-signed release may be digitally attached to the client's digital records for further use and/or access, such as directly on the first user device, a cloud network, and/or other location. In certain embodiments, the application may enable artist billing to be conducted via the application, where clients may be able to submit payment for services rendered by the artist. In certain embodiments, the billing may account for a time of the session, flat rate arrangements, materials used, and/or other measurements. In certain embodiments, the application may facilitate the setting of presets for machine usage based on tattoo artist requirements and/or preferences, and the ability to load the presets via voice or direct screen interactions with the application.

The system 100 may also enable for the scanning of bar codes, quick response codes, and/or other codes associated with tattoo equipment and/or suppose so that corresponding item may be tracked and/or usage of the item may be tracked. In certain embodiments, the application may facilitate tracking and replenishment of expendables, such as tattoo needles, ink, ink cups, anti-contamination tools, and/or supplies. In certain embodiments, the tracked information may be utilized for information purposes, reordering supplies, billing to clients, and/or tracking for artists working as independent contractors for usage of studio supplies. In certain embodiments, the application and system 100 may be utilized to book tattoo sessions and/or schedule any type of appointment and/or consultation. The application may also do automatic calendaring for any appointments made via the application, which may be utilized by tattoo artists and/or the tattoo clients. In certain embodiments, the first user device 102, such as via the application, may be configured to record media content associated with a tattoo session, take photographs associated with a tattoo session or tattoo, session activities, expendables utilized by tattoo machines 120 and/or tattoo artists, session times, and device usage statistics. In certain embodiments, the system 100 and application may be utilized to select images and/or media content accessible to the application to be posted directly to social media applications, websites, and/or other online resources. in certain embodiments, the application may be utilized to send out emails to clients, stakeholders, and/or management. In further embodiments, the system 100 and application may be utilized to track machine usage information for the purposes of manufacturer warranty support and conduct machine trouble flagging, such as potential mechanical issues. If a problem with a machine or component is detected, the application may transmit an alert via the application to a tattoo artist or other individual identifying the nature of the problem so that it may be rectified.

In certain embodiments, the system 100 and application may allow for the customization of the information displayed via the user interface of the application, the layout of information displayed on the user interface, and formats for tattoo machines, such as those with LCD displays. In certain embodiments, the system 100 and application may be utilized to customize the behaviors and meanings of LEDs disposed on the tattoo machine 120 and/or tattoo machine accessories. For example, the LEDs may be customized to flash in a certain manner, display a certain color, or output various patterns of light to alert the user of a particular condition associated with the tattoo machines 120 and/or tattoo machine accessories. In certain embodiments, in addition to electronic give settings and voltage settings that can be set via the application, the application may be utilized to transmit signals to adjust the needle in a needle cartridge of the tattoo machine 120. For example, the application may be utilized to adjust the position of the needle or even replace the needle with a new needle or adjust the needle length that protrudes from the needle cartridge. In certain embodiments, the application may be utilized to adjust functionality of sensors of each of the components to the extent that sensors may be utilized with the components (e.g. LEDs, pressure sensors, temperature sensors (components are overheating), motion sensors, any types of sensors), humidity/moisture sensors (e.g. to allow both monitoring and fail-safe to automatically turn the power off for a device when the machine is detected to be ‘wet’.

In certain embodiments, the application may incorporate any demographic information for the user in the user's user profiles. In certain embodiments, over time, the application can learn the user's preferences and settings and automatically save profiles to include certain settings. In certain embodiments, certain types of tattoos may require a certain set of settings and other types of tattoos may require other settings. The information associated with such settings may also be stored in the user profiles for easy access and deployment. In certain embodiments, the settings for the components and/or devices can also auto-adjust in real time based on the user profile of a user loaded into the application. In certain embodiments, the application may enable settings to load for tattoo machines 120 and/or accessories simply by inputting the user's name that is using the tattoo machine 120 or potentially based on the person receiving the tattoo. In certain embodiments, the system 100 and application may be configured to receive feedback from individuals receiving tattoos and/or tattoo artists. Additionally, feedback may be received into the application via a social media application communicatively linked to the social media application. In certain embodiments, session information and media content taken during a session may be posted to such social media applications via the application as well. In certain embodiments, a tattoo session may be live-streamed using the application and/or to a social media application. In certain embodiments, the application and system 100 may provide the ability to change the functionality of buttons on the components of the tattoo machine 120 and/or tattoo machine accessories. For example, certain user may want to have different functionality for a button based on its location on the component.

In certain embodiments, the system 100 and application may enable security settings and/or multi-factor authentication so that only authorized individuals can use the tattoo machine, the application, and/or its components. In certain embodiments, the system 100 and application may enable loading of an image (or other media content) of a tattoo to be tattooed on an individual into the application for reference by the tattoo artist and/or the tattoo client. In certain embodiments, the image (or other media content) may be shown on a user interface of the tattoo machine 120 and/or tattoo machine accessories themselves. In certain embodiments, the system 100 and application may be configured to auto-detect the voice of the user and load the associated user's profile and settings based on the detection of the voice of the user. Additionally, the system 100 and application may enable the user to activate or deactivate voice commands from a list. In certain embodiments, the system 100 and application may enable the user to create new voice commands or merge existing voice commands depending on desired user functionality (or submit a request for a new voice command via the application to the developers of the application). In certain embodiments, the system and application may be configured to detect a voice of a user to authenticate the user for access to the tattoo machine, the application, and/or tattoo machine accessories. In certain embodiments, the system 100 and/or application may be configure to record and store custom commands created by the user as well.

In further embodiments, the system 100 and application may be configured to enable the devices to be paired and/or communicatively coupled using any communications technologies and/or protocols, such as, but not limited to, internet-of-things-based protocols, short-range protocols, mid-range protocols, and/or long-range protocols. In certain embodiments, devices may be paired by scanning and detecting bar codes, quick responses codes, any visual code, audio tones, video content, any type of identifier, or a combination thereof. For example, a camera of the first user device 120 may be utilized to scan such identifiers. In certain embodiments, components of the devices of the system 100, the tattoo machine 120, and/or the tattoo machine accessories can share component status information (e.g. battery information, heat information, speed information, orientation information, component-life information, etc.) with the application and other components and/or devices that they are paired with. In certain embodiments, the system 100 and application may be configured to share information with remote devices as well for further analyzing and/or processing and/or functionality, such as by sharing information with servers 140, 145, 150, 160. For example, information that may be shared may include, but is not limited to, tattoo session run times, machine settings, such as run voltage, electronic give levels, battery level information, and/or component information. In further embodiments, the tattoo machine 120, application, and/or tattoo machine accessories may be configured to communicate with and control other devices, such as smart lights (perhaps the tattoo artist would like to adjust lighting depending on the art or individual receiving the tattoo), computers, cleaning supply devices (e.g. tattoo machine can cause an electronic dispenser to dispense wipes or other cleaning supplies), and/or a device associated with a receptionist/manager of the business (notify the receptionist when the next client can come in or estimated wait time to see a tattoo artist).

In certain embodiments, the system 100 and application may enable other users to provide feedback and/or opinions on another user's tattoo and/or a tattoo artist's work. In certain embodiments, the system 100 and application may provide feedback, such as via a connected social media application, to a user or tattoo artist regarding potential modifications to the tattoo via the application. In certain embodiments, the system 100 and application may incorporate artificial intelligence and/or machine learning capabilities. For example, as data about the components is gathered over time, the software powering the application (or other software) can adjust a user's selected or loaded settings to a more optimal set of settings. As an example, perhaps a user typically uses a preferred set of settings, but regularly changes some of the settings during the middle of a tattoo session. If the system 100 and/or application learns that a tattoo artist regularly performs this change in settings during a tattoo session, the system 100 and/or application can suggest to the user a modified set of settings which may include these changes at the beginning of a tattoo session or at another selected time. In certain embodiments, the system 100 and/or application can learn over time that customers with certain demographic details require different tattoo machine settings and the system 100 and/or application may automatically suggest different settings based on the demographics information of the individual to be tattooed and/or the tattoo artist. In certain embodiments, the system 100 and/or application can learn over time which component failures lead to complete inability to use the tattoo machine 1200 and/or tattoo machine accessories and order these components more frequently. The system 100 and/or application may have the ability to monitor full machine and internal component performance quality to determine which devices are better than others and/or which supplies are better than others. Over time, the system 100 and/or application can gather feedback from tattoo artists, individuals receiving tattoos, or third parties to adjust the user interface, adjust settings, adjust possible tattoos, and/or adjust tattoo options to be presented to users. For example, if the system 100 and/or application determines that a certain user likes car tattoos, the system 100 and/or application may suggest other car tattoos or tattoos that have some relation to cars for the user to get on a subsequent tattoo session. Additionally, the system 100 and/or application may be configured to upload artwork of a tattoo created by a tattoo artist so that a stencil corresponding to the artwork may be printed using the application and transferred to the body of a client.

In certain embodiments, the first user device 102, such as via the application, may be utilized to scan images and/or media content directly into the application for further use. In certain embodiments, the system 100 and/or application may enable images and/or media content to be downloaded online resources onto the application and first user device 102. The application may enable a user to edit and scale such content, such as by performing crops, color adjustments, image mergers, any other image edits, or a combination thereof. Once the images and/or media content are finalized, the end result may be transmitted to a printer for printing by the application. The printer may print a stencil at a size and format desired by the artist and/or tattoo recipient. In certain embodiments, the original media content may be displayed on the phone screen during a tattoo session and the application may allow switching between the machine settings and the artwork displayed during the session. In certain embodiments, the application may be utilized to project the artwork on a secondary screen, such as a TV or other display, during a tattoo session. The system 100 and application may also enabling sharing of the artwork with clients and/or social media applications as well.

In certain embodiments, the system 100 and application provided in the present disclosure may also be utilized to determine signal information between various devices of the system 100. For example, the system 100 and application may be configured to determine signal information between the first user device 102 and the tattoo machine 120. Signal information may include, but is not limited to, the type of signal between two or more devices, the signal strength between or among two or more device, whether another signal is interfering with a signal, the amplitude of the signal, the frequency of the signal, whether the signal is degrading or amplifying, any other signal information, or a combination thereof. The signal information may be displayed on the user interface of the first user device 102, on the tattoo machine 120, the wireless battery pack 122, the hover device 124, any other device of the system 100, or a combination thereof. The signal information may be displayed next to an icon of a wireless signal that shows the strength of the signal in bars (or other desired format). In an exemplary scenario, a signal level>=−50 may indicate a very strong and good signal (shown by 4 bars on the user interface); a signal level<=−51 && level>=−67 may indicate an acceptable signal (shown by 3 bars on the user interface); a signal level<=−68 && level>=−80 may indicate a bad or poor signal (shown by 2 bars on the user interface); a signal level<=−81 && level>=−96 may indicate a very bad or very poor signal (shown by 1 bar on the user interface; and no connection (disconnection) may be indicated by 0 bars on the user interface. In certain embodiments, in the even there is a disconnection, the user interface may indicate the disconnected status, and, for a while, may allow for reconnection of a device(s). In the event that no reconnection occurs within a particular time period, the user interface may revert back to a home screen or other screen. In certain embodiments, any of the devices may measure the signal information on a periodic basis, a continuous basis, at selected times, and/or at random times.

The systems and methods disclosed herein may include still further functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and the various methods discloses herein. For example, by training the system 100 over time based on data and/or other information provided and/or generated in the system 100, a reduced amount of computer operations may need to be performed by the devices in the system 100 using the processors and memories of the system 100 than compared to traditional methodologies. In such a context, less processing power needs to be utilized because the processors and memories do not need to be dedicated for processing. As a result, there are substantial savings in the usage of computer resources by utilizing the software, techniques, and algorithms provided in the present disclosure. In certain embodiments, various operative functionality of the system 100 may be configured to execute on one or more graphics processors and/or application specific integrated processors.

Notably, in certain embodiments, various functions and features of the system 100 and methods may operate without any human intervention and may be conducted entirely by computing devices. In certain embodiments, for example, numerous computing devices may interact with devices of the system 100 to provide the functionality supported by the system 100. Additionally, in certain embodiments, the computing devices of the system 100 may operate continuously and without human intervention to reduce the possibility of errors being introduced into the system 100. In certain embodiments, the system 100 and methods may also provide effective computing resource management by utilizing the features and functions described in the present disclosure. For example, in certain embodiments, devices in the system 100 may transmit signals indicating that only a specific quantity of computer processor resources (e.g. processor clock cycles, processor speed, etc.) may be devoted to adjusting voltage settings, adjusting e-give settings, monitoring the tattoo machines and/or tattoo machine accessories, and/or performing any other operation conducted by the system 100, or any combination thereof. For example, the signal may indicate a number of processor cycles of a processor may be utilized to increase or decrease the voltage of the tattoo machine, and/or specify a selected amount of processing power that may be dedicated to generating or any of the operations performed by the system 100. In certain embodiments, a signal indicating the specific amount of computer processor resources or computer memory resources to be utilized for performing an operation of the system 100 may be transmitted from the first and/or second user devices 102, 111 to the various components of the system 100. In certain embodiments, the devices in the system 100 may transmit signals indicating that only a specific amount of battery power may be supplied to devices connected to the hover device 124 and/or the wireless battery pack 122.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected value. In certain embodiments, the system 100 and methods may include transmitting signals to the memory devices utilized in the system 100, which indicate which specific sections of the memory should be utilized to store any of the data utilized or generated by the system 100. Notably, the signals transmitted to the processors and memories may be utilized to optimize the usage of computing resources while executing the operations conducted by the system 100. As a result, such functionality provides substantial operational efficiencies and improvements over existing technologies.

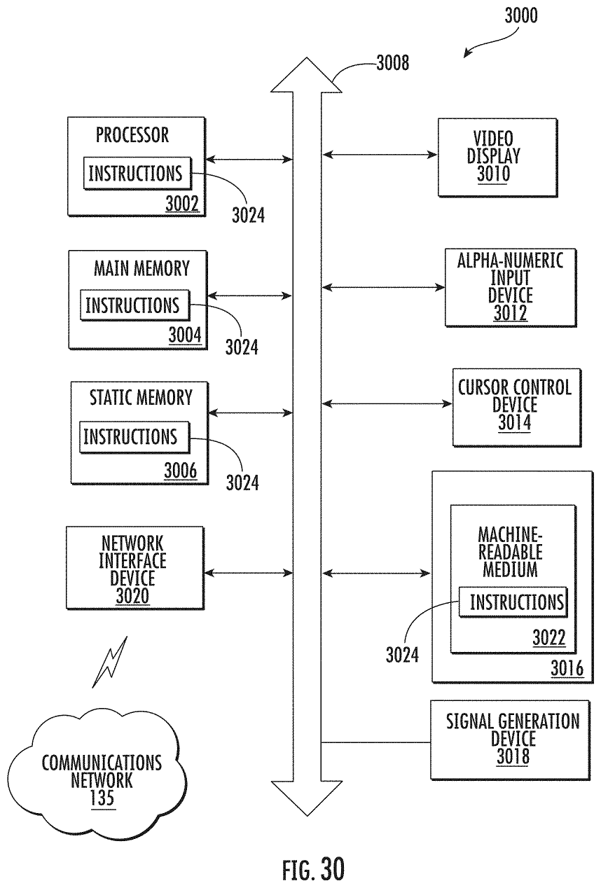

Referring now also to FIG. 30, at least a portion of the methodologies and techniques described with respect to the exemplary embodiments of the system 100 can incorporate a machine, such as, but not limited to, computer system 30, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100. As another example, the computer system 3000 may assist with adjusting voltages for the tattoo machine, accessories of the tattoo machine (e.g. the hover device 124, the footswitch 126, the wireless battery pack 122, etc.), or a combination thereof. As a further example, the computer system 3000 may also assist with pairing and unpairing the tattoo machine with the first user device 102, the second user device 111, the hover device 124, the footswitch 126, the wireless battery pack 122, any other accessory, any device and/or program of the system 100, or a combination thereof.

In some embodiments, the machine may operate as a standalone device. In some embodiments, the machine may be connected (e.g., using communications network 135, another network, or a combination thereof) to and assist with operations performed by other machines and systems, such as, but not limited to, the first user device 102, the second user device 111, the tattoo machine 120, the wireless battery pack 122, the hover device 124, the footswitch 126, the server 140, the server 145, the server 150, the database 155, the server 160, any other system, program, and/or device, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 3000 may include a processor 3002 (e.g., a central processing unit (CPU), a graphics processing unit (GPU, or both), a main memory 3004 and a static memory 3006, which communicate with each other via a bus 3008. The computer system 3000 may further include a video display unit 3010, which may be, but is not limited to, a liquid crystal display (LCD), a flat panel, a solid state display, or a cathode ray tube (CRT). The computer system 3000 may include an input device 3012, such as, but not limited to, a keyboard, a cursor control device 3014, such as, but not limited to, a mouse, a disk drive unit 3016, a signal generation device 3018, such as, but not limited to, a speaker or remote control, and a network interface device 3020.

The disk drive unit 3016 may include a machine-readable medium 3022 on which is stored one or more sets of instructions 3024, such as, but not limited to, software embodying any one or more of the methodologies or functions described herein, including those methods illustrated above. The instructions 3024 may also reside, completely or at least partially, within the main memory 3004, the static memory 3006, or within the processor 3002, or a combination thereof, during execution thereof by the computer system 3000. The main memory 3004 and the processor 3002 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 3022 containing instructions 3024 so that a device connected to the communications network 135, another network, or a combination thereof, can send or receive voice, video or data, and communicate over the communications network 135, another network, or a combination thereof, using the instructions. The instructions 3024 may further be transmitted or received over the communications network 135, another network, or a combination thereof, via the network interface device 3020.

While the machine-readable medium 3022 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A system, comprising:
a memory that stores instructions; and
a processor that executes the instructions to perform operations, the operations comprising:
providing, via a user interface of an application, an option to scan for devices within a communication range of a device utilizing the application, wherein the devices comprise a tattoo machine, a tattoo machine accessory, or a combination thereof;
scanning, after the option is selected, for the devices within the communication range of the device utilizing the application;
detecting, based on the scanning, the tattoo machine, the tattoo machine accessory, or both, within the communication range of the device utilizing the application;
pairing the device with the tattoo machine, the tattoo machine accessory, or both; and
enabling, after pairing the device with the tattoo machine, the tattoo machine accessory, or both, the device to control the tattoo machine, the tattoo machine accessory or both, by utilizing the application.

2. The system of claim 1, wherein the operations further comprise adjusting a setting of the tattoo machine, the tattoo machine accessory, or both, by utilizing the application.

3. The system of claim 2, wherein the setting comprises a voltage setting, a power state, an electronic give setting, a needle adjustment setting, a sensor setting, a component setting, any setting, or a combination thereof.

4. The system of claim 1, wherein the operations further comprise enabling the device to control the tattoo machine, the tattoo machine accessory, or both, by utilizing a voice command.

5. The system of claim 1, wherein the operations further comprise facilitating the pairing of the device with the tattoo machine, the tattoo machine accessory, or both, by utilizing Bluetooth™, Zigbee™, Z-wave™, near-field communication technology, radio frequency technology, a short-range wireless protocol, a long-range wireless protocol, wireless technology, an internet of things-based protocol, a quick response-based technology, a barcode technology, or a combination thereof.

6. The system of claim 1, wherein the operations further comprise facilitating pairing of the tattoo machine with the tattoo machine accessory.

7. The system of claim 1, wherein the tattoo machine accessory comprises a wireless battery power supply, a gesture-based power supply, any other accessory, or a combination thereof.

8. The system of claim 1, wherein the operations further comprise activating or deactivating the tattoo machine, the tattoo machine accessory, or both, by utilizing the application.

9. The system of claim 1, wherein the operations further comprise providing access to a tutorial for utilizing the tattoo machine, the tattoo machine accessory, or both, and providing a plurality of help options for assisting a user of the application.

10. The system of claim 1, wherein the operations further comprise providing, via the application, a release authorization for a recipient of a tattoo to electronically sign via the application, a digital session timer for timing a tattoo session conducted using the tattoo machine, a payment portal for paying for tattoo services or products, default settings for the tattoo machine, default settings for the tattoo machine accessory, a tattoo session booking capability, a product ordering portal, or a combination thereof.

11. The system of claim 1, wherein the operations further comprise tracking status information for components of the tattoo machine, the tattoo machine accessory, or both, and wherein the operations further comprise tracking usage statistics for the tattoo machine, the tattoo machine accessory, or both.

12. The system of claim 1, wherein the operations further comprise capturing media content associated with a tattoo session for a tattoo recipient, and wherein the operations further comprise enabling the media content to be accessed via the application.

13. The system of claim 1, wherein the operations further comprise detecting an occurrence of a problem associated with the tattoo machine, the tattoo machine accessory, or both, and wherein the operations further comprise generating a report including an identification of the occurrence of the problem.

14. A method, comprising:
providing, via a user interface of an application and by utilizing instructions from a memory that are executed by a processor, an option to scan for devices within a communication range of a device utilizing the application, wherein the devices comprise a tattoo machine, a tattoo machine accessory, or a combination thereof;
scanning, after the option is selected, for the devices within the communication range of the device utilizing the application;
detecting, based on the scanning, the tattoo machine, the tattoo machine accessory, or both, within the communication range of the device utilizing the application;
pairing the device with the tattoo machine, the tattoo machine accessory, or both; and
enabling, after pairing the device with the tattoo machine, the tattoo machine accessory, or both, the device to control the tattoo machine, the tattoo machine accessory or both, by utilizing the application.

15. The method of claim 14, further comprising adjusting a behavior of a sensor of the tattoo machine, the tattoo machine accessory or both, via the application, wherein the sensor comprises a pressure sensor, a motion sensor, a light emitting diode, a temperature sensor, any type of sensor, or a combination thereof.

16. The method of claim 14, further comprising enabling creation of a user account to access functionality of the application.

17. The method of claim 14, further comprising uploading media content into the application, wherein the media content comprises artwork associated with a tattoo to be tattooed onto a recipient, a drawing, video content, audio content, augmented reality content, virtual reality content, or a combination thereof.

18. The method of claim 17, further comprising enabling the media content to be shared via the application to a social media application, a email address, an instant message, a text message, a website, any digital resource, or a combination thereof.

19. The method of claim 14, further comprising unpairing the device from the tattoo machine, the tattoo machine accessory, or both.

20. A computer-readable device comprising instructions, which, when loaded and executed by a processor, cause the processor to perform operations, the operations comprising:
providing, via a user interface of an application, an option to scan for devices within a communication range of a device utilizing the application, wherein the devices comprise a tattoo machine, a tattoo machine accessory, or a combination thereof;
scanning, after the option is selected, for the devices within the communication range of the device utilizing the application;
detecting, based on the scanning, the tattoo machine, the tattoo machine accessory, or both, within the communication range of the device utilizing the application;
pairing the device with the tattoo machine, the tattoo machine accessory, or both; and
enabling, after pairing the device with the tattoo machine, the tattoo machine accessory, or both, the device to control the tattoo machine, the tattoo machine accessory or both, by utilizing the application.
